SEQUENCE LISTING (1 sequence)

SEQ ID NO: 1 - protein, 26 aa, Artificial Sequence
  Chemically synthesized
CDELPQLVTLPHPNLHGPEELDVPST

(19) United States
(12) Patent Application Publication
(10) Pub. No.: US 2007/0060608 A1
(43) Pub. Date: Mar. 15, 2007

(54) Combination products with carboxylic acid derivatives that inhibit the binding of integrins to their receptors and other therapeutic compounds

(75) Inventors: Peter Vanderslice, Houston, TX (US); George Holland, Houston, TX (US); Neng Yang Shih, North Caldwell, NJ (US); Robert G. Asianian, Rockaway, NJ (US); Richard W. Chapman, Hillsboro, NJ (US); William Kreutner, West Paterson, NJ (US)

Correspondence Address: WOOD, PHILLIPS, KATZ, CLARK & MORTIMER, 500 W. MADISON STREET, SUITE 3800, CHICAGO, IL, 60661, US

(73) Assignees: ENCYSIVE PHARMACEUTICALS INC., Houston, TX (US); SCHERING CORPORATION, Kenilworth, NJ (US)

(21) Appl. No.: 10/533,693

(22) PCT Filed: Nov. 7, 2003
(86) PCT No.: PCT/US03/35526

§ 371 Date: Dec. 14, 2005

Related U.S. Application Data
(60) Provisional application No. 60/424,928, filed on Nov. 8, 2002.

(51) Int. Cl. A61K 31/4704; A61K 31/4412

(52) U.S. Cl. 514/309; 514/345; 546/143; 546/291

(57) ABSTRACT

A composition, method and kit comprising a compound for the inhibition of the binding of α₄β₁ integrin to its receptors, for example VCAM-1 (vascular cell adhesion molecule-1) and fibronectin and other therapeutic compounds for the control or prevention of diseases states in which α₄β₁ is involved.

### FIELD OF THE INVENTION

This invention is directed generally to combination products of and methods of co-administering compounds that inhibit the binding of α₄β₁ integrin to its receptors, for example VCAM-1 (vascular cell adhesion molecule-1) and fibronectin and other therapeutic compounds. The invention also relates to the use of such compositions and methods for the control or prevention of disease states in which α₄β₁ is involved.

### BACKGROUND OF THE INVENTION

When a tissue has been invaded by a microorganism or has been damaged, white blood cells, also called leukocytes, play a major role in the inflammatory response. One of the most important aspects of the inflammatory response involves the cell adhesion event. Generally, white blood cells are found circulating through the bloodstream. However, when a tissue is infected or becomes damaged, the white blood cells recognize the invaded or damaged tissue, bind to the wall of the capillary and migrate through the capillary into the affected tissue. These events are mediated by a family of proteins called cell adhesion molecules.

There are three main types of white blood cells: granulocytes, monocytes and lymphocytes. The integrin α₄β₁ (also called VLA-4 for very late antigen-4) is a heterodimeric protein expressed on the surface of monocytes, lymphocytes and two subclasses of granulocytes: eosinophils and basophils. This protein plays a key role in cell adhesion through its ability to recognize and bind VCAM-1 and fibronectin, proteins associated with the endothelial cells that line the interior wall of capillaries.

Following infection or damage of tissue surrounding a capillary, endothelial cells express a series of adhesion molecules, including VCAM-1, that are critical for binding the white blood cells that are necessary for fighting infection. Prior to binding to VCAM-1 or fibronectin, the white blood cells initially bind to certain adhesion molecules to slow their flow and allow the cells to “roll” along the activated endothelium. Monocytes, lymphocytes, basophils and eosinophils are then able to firmly bind to VCAM-1 or fibronectin on the blood vessel wall via the α₄β₁ integrin. There is evidence that such interactions are also involved in transmigration of these white blood cells into the damaged tissue as well as the initial rolling event itself.

Although white blood cell migration to the site of injury helps fight infection and destroy foreign material, in many instances this migration can become uncontrolled, with white blood cells flooding to the scene, causing widespread tissue damage. Compounds capable of blocking this process, therefore, may be beneficial as therapeutic agents. Thus, it would be useful to develop inhibitors that would prevent the binding of white blood cells to VCAM-1 and fibronectin.

Some of the diseases that might be treated by the combination of compounds for the inhibition of α₄β₁ binding and other therapeutic compounds include, but are not limited to, psoriasis, atherosclerosis, rheumatoid arthritis, asthma, allergy, multiple sclerosis, Guillan-Barr Syndrome, lupus, inflammatory bowel disease, graft rejection, contact hypersensitivity, reperfusion injury and type I diabetes.

It is therefore an object of the invention to provide novel compositions comprising compounds which are inhibitors of α₄β₁ binding and other therapeutic agents.

### BRIEF SUMMARY OF THE INVENTION

The present invention is directed to pharmaceutical compositions that comprise a compound that inhibits binding of integrins to their receptors and one or more other therapeutic agents.

More particularly, the present invention is directed to a pharmaceutical composition that comprises a compound of Formula I 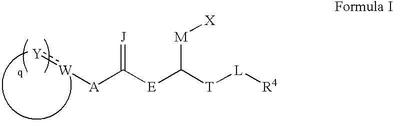
wherein Y, at each occurrence, is independently selected from the group consisting of C(O), N, CR¹, C(R²)(R³), NR⁵, CH, O and S;
q is an integer of from 3 to 10;
A is selected from the group consisting of O, S, C(R¹⁶)(R¹⁷) and NR⁶;
E is selected from the group consisting of CH₂, O, S, and NR⁷;
J is selected from the group consisting of O, S and NR⁸;
T is selected from the group consisting of C(O) and (CH₂)_{b} wherein b is an integer of from 0 to 3;
M is selected from the group consisting of C(R⁹)(R¹⁰) and (CH₂)ᵤ, wherein u is an integer of from 0 to 3;
L is selected from the group consisting of O, NR¹¹, S, and (CH₂)ₙ wherein n is an integer of 0 or 1;
X is selected from the group consisting of CO₂B, PO₃H₂, SO₃H, SO₂NH₂, SO₂NHCOR¹², OPO₃H₂, C(O)NHC(O)R¹³, C(O)NHSO₂R¹⁴, hydroxyl, tetrazolyl and hydrogen;
W is selected from the group consisting of C, CR¹⁵ and N;
B is selected from the group consisting of hydrogen, alkyl, alkenyl, alkynyl, hydroxyalkyl, haloalkyl, —CF₃, cycloalkyl, cycloalkenyl, cycloalkynyl, cycloalkylalkyl, aryl, heterocyclyl, alkylaryl, aralkenyl, aralkyl, alkylheterocyclyl, heterocyclylalkyl and aryloxyalkyl; and
R¹, R², R³, R⁴, R⁵, R⁶, R⁷, R⁸, R⁹, R¹⁰, R¹¹, R¹², R¹³, R¹⁴, R¹⁵, R¹⁶ and R¹⁷ at each occurrence are independently selected from the group consisting of hydrogen, halogen, alkyl, alkenyl, alkynyl, alkoxy, alkenoxy, alkynoxy, thioalkoxy, hydroxyalkyl, aliphatic acyl, —CF₃, —CO₂H, —SH, —CN, —NO₂, —NH₂, —OH, alkynylamino, alkoxycarbonyl, heterocyclyl, carboxy, —N(C₁-C₃ alkyl)-C(O)(C₁-C₃ alkyl), —NHC(O)N(C₁-C₃ alkyl)C(O)—NH(C₁-C₃ alkyl), —NHC(O)NH(C₁-C₆ alkyl), —NHSO₂(C₁-C₃ alkyl), —NHSO₂(aryl), alkoxyalkyl, alkylamino, alkenylamino, di(C₁-C₃)amino, —C(O)O—(C₁-C₃)alkyl, —C(O)NH—(C₁-C₃)alkyl, —C(O)N(C₁-C₃ alkyl)₂, —CH═NOH, —PO₃H₂, —OPO₃H₂, haloalkyl, alkoxyalkoxy, carboxaldehyde, carboxamide, cycloalkyl, cycloalkenyl, cycloalkynyl, cycloalkylalkyl, aryl, aroyl, aryloxy, arylamino, biaryl, thioaryl, diarylamino, heterocyclyl, alkylaryl, aralkenyl, aralkyl, alkylheterocyclyl, heterocyclylalkyl, sulfonyl, —SO₂—(C₁-C₃ alkyl), —SO₃—(C₁-C₃ alkyl), sulfonamido, carbamate, aryloxyalkyl and —C(O)NH(benzyl) groups;
   wherein B, R¹, R², R³, R⁴, R⁵, R⁶, R⁷, R⁸, R⁹, R¹⁰, R¹¹, R¹², R¹³, R¹⁴, R¹⁵, R¹⁶ and R¹⁷ are unsubstituted or substituted with at least one electron donating or electron withdrawing group;
   wherein when L is NR¹¹, R⁴ and R¹¹ taken together may form a ring;
   and wherein when M is C(R⁹)(R¹⁰), R⁹ and R¹⁰ taken together may form a ring;
   and wherein when A is NR⁶ and at least one Y is CR¹, R¹ and R⁶ taken together may form a ring;

or a pharmaceutically acceptable salt thereof;

and one or more other therapeutic compounds.

For Formula I, presently preferred compounds may have A as NR⁶; E as NR⁷; J as O; M as C(R⁹)(R¹⁰); q as 4 or 5; T as (CH₂)_{b} wherein b is 0; L as (CH₂)ₙ wherein n is 0; X as CO₂B; W as C or CR¹⁵; R⁴ as aryl, alkylaryl, aralkyl, heterocyclyl, alkylheterocyclyl or heterocyclylalkyl; and R⁶, R⁷, R⁹, R¹⁰ and R¹⁵ independently as hydrogen or lower alkyl.

More specifically, the compositions of this invention comprise compounds of Formula II 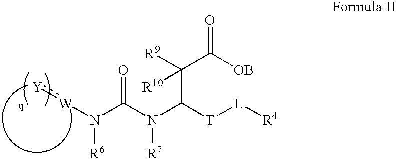
wherein Y, at each occurrence, is independently selected from the group consisting of C(O), N, CR¹, C(R²)(R³), NR⁵, CH, O and S;
q is an integer of from 3 to 7;
T is selected from the group consisting of C(O) and (CH₂)_{b} wherein b is an integer of 0 to 3;
L is selected from the group consisting of O, NR¹¹, S, and (CH₂), wherein n is an integer of 0 or 1;
W is selected from the group consisting of C, CR¹⁵ and N;
B is selected from the group consisting of hydrogen, alkyl, alkenyl, alkynyl, hydroxyalkyl, haloalkyl, —CF₃, cycloalkyl, cycloalkenyl, cycloalkynyl, cycloalkylalkyl, aryl, heterocyclyl, alkylaryl, aralkenyl, aralkyl, alkylheterocyclyl, heterocyclylalkyl and aryloxyalkyl; and
R¹, R², R³, R⁴, R⁵, R⁶, R⁷, R⁹, R¹⁰, R¹¹ and R¹⁵ are independently selected from the group consisting of hydrogen, halogen, alkyl, alkenyl, alkynyl, alkoxy, alkenoxy, alkynoxy, thioalkoxy, hydroxyalkyl, aliphatic acyl, —CF₃, —CO₂H, —SH, —CN, —NO₂, —NH₂, —OH, alkynylamino, alkoxycarbonyl, heterocycloyl, carboxy, —N(C₁-C₃ alkyl)-C(O)(C₁-C₃ alkyl), —NHC(O)N(C₁-C₃ alkyl)C(O)NH(C₁-C₃alkyl), —NHC(O)NH(C₁-C₆ alkyl), —NHSO₂(C₁-C₃ alkyl), —NHSO₂(aryl), alkoxyalkyl, alkylamino, alkenylamino, di(C₁-C₃)amino, —C(O)O—(C₁-C₃)alkyl, C(O)NH—(C₁-C₃)alkyl, —C(O)N(C₁-C₃ alkyl)₂, —CH═NOH, —PO₃H₂, —OPO₃H₂, haloalkyl, alkoxyalkoxy, carboxaldehyde, carboxamide, cycloalkyl, cycloalkenyl, cycloalkynyl, cycloalkylalkyl, aryl, aroyl, aryloxy, arylamino, biaryl, thioaryl, diarylamino, heterocyclyl, alkylaryl, aralkenyl, aralkyl, alkylheterocyclyl, heterocyclylalkyl, sulfonyl, —SO₂—(C₁-C₃ alkyl), —SO₃—(C₁-C₃ alkyl), sulfonamido, carbamate, aryloxyalkyl and —C(O)NH(benzyl) groups;
   wherein B, R¹, R², R³, R⁴, R⁵, R⁶, R⁷, R⁹, R¹⁰, R¹¹ and R¹⁵ are unsubstituted or substituted with at least one electron donating or electron withdrawing group;
   wherein when L is NR¹¹, R⁴ and R¹¹ taken together may form a ring;
      and wherein R⁹ and R¹⁰ taken together may form a ring;
      and wherein when at least one Y is CR¹, R¹ and R⁶ taken together may form a ring;

or a pharmaceutically acceptable salt thereof and one or more other therapeutic compounds.

For Formula II, presently preferred compounds may have q as 4 or 5; W as C or CR¹⁵; T as (CH₂)_{b} wherein b is 0; L as (CH₂)ₙ wherein n is 0; R⁴ as aryl, alkylaryl, aralkyl, heterocyclyl, alkylheterocyclyl or heterocyclylalkyl; and R⁶, R⁷, R⁹, R¹⁰ and R¹⁵ as independently hydrogen or lower alkyl.

Even more specifically, the compositions of this invention comprise a compound of Formula III 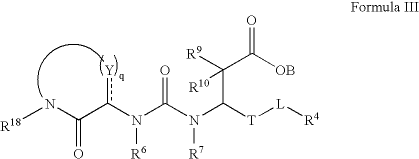
wherein Y, at each occurrence, is independently selected from the group consisting of C(O), N, CR¹, C(R² )(R³), NR5, CH, O and S;
q is an integer of from 2 to 5;
T is selected from the group consisting of C(O) and (CH₂)_{b} wherein b is an integer of 0 to 3;
L is selected from the group consisting of O, NR¹¹, S, and (CH₂)ₙ wherein n is an integer of 0 or 1;
R⁵, R⁶, R⁷, R¹¹ and R¹⁸ are each independently selected from the group consisting of alkyl, alkenyl, alkynyl, hydroxyalkyl, aliphatic acyl, alkynylamino, alkoxycarbonyl, heterocycloyl, —CH═NOH, haloalkyl, alkoxyalkoxy, carboxaldehyde, carboxamide, cycloalkyl, cycloalkenyl, cycloalkynyl, cycloalkylalkyl, aryl, aroyl, aryloxy, arylamino, biaryl, thioaryl, diarylamino, heterocyclyl, alkylaryl, aralkenyl, aralkyl, alkylheterocyclyl, heterocyclylalkyl, carbamate, aryloxyalkyl, hydrogen and—C(O)NH(benzyl) groups;
B is selected from the group consisting of hydrogen, alkyl, alkenyl, alkynyl, hydroxyalkyl, haloalkyl, —CF₃, cycloalkyl, cycloalkenyl, cycloalkynyl, cycloalkylalkyl, aryl, heterocyclyl, alkylaryl, aralkenyl, aralkyl, alkylheterocyclyl, heterocyclylalkyl and aryloxyalkyl; and
R¹, R², R³, R⁴, R⁹ and R¹⁰ are independently selected from the group consisting of hydrogen, halogen, alkyl, alkenyl, alkynyl, alkoxy, alkenoxy, alkynoxy, thioalkoxy, hydroxyalkyl, aliphatic acyl, —CF₃, —CO₂H, —SH, —CN, —NO₂, —NH₂, —OH, alkynylamino, alkoxycarbonyl, heterocycloyl, carboxy, —N(C₁-C₃ alkyl)-C(O)(C₁-C₃ alkyl), —NHC(O)N(C₁-C₃ alkyl)C(O)NH(C₁-C₃ alkyl), —NHC(O)NH(C₁-C₆ alkyl), —NHSO₂(C₁-C₃ alkyl), —NHSO₂(aryl), alkoxyalkyl, alkylamino, alkenylamino, di(C₁-C₃)amino, —C(O)O—(C₁-C₃)alkyl, —C(O)NH—(C₁-C₃)alkyl, —C(O)N(C₁-C₃ alkyl)₂, —CH═NOH, —PO₃H₂, —OPO₃H₂, haloalkyl, alkoxyalkoxy, carboxaldehyde, carboxamide, cycloalkyl, cycloalkenyl, cycloalkynyl, cycloalkylalkyl, aryl, aroyl, aryloxy, arylamino, biaryl, thioaryl, diarylamino, heterocyclyl, alkylaryl, aralkenyl, aralkyl, alkylheterocyclyl, heterocyclylalkyl, sulfonyl, —SO₂—(C₁-C₃ alkyl), —SO₃—(C₁-C₃ alkyl), sulfonamido, carbamate, aryloxyalkyl and —C(O)NH(benzyl) groups;
wherein B, R¹, R², R³, R⁴, R⁵, R⁶, R⁷, R⁹, R¹⁰, R¹¹ and R¹⁸ are unsubstituted or substituted with at least one electron donating or electron withdrawing group;
wherein when L is NR¹¹, R⁴ and R¹¹ taken together may form a ring;
   and wherein R⁹ and R¹⁰ taken together may form a ring;
   and wherein when at least one Y is CR¹, R¹ and R⁶ taken together may form a ring;

or a pharmaceutically acceptable salt thereof and one or more other therapeutic compounds.

Presently preferred compounds of Formula III may have R¹⁸ as hydrogen, alkyl, aryl, aralkyl, cycloalkyl, alkylheterocyclyl, heterocyclylalkyl or heterocyclyl; T as (CH₂)_{b} wherein b is 0; L as (CH₂)ₙ wherein n is 0; Y as CR¹ and C(R²)(R³) and q as 2 or 3.

In Formula III, the portion of the molecule

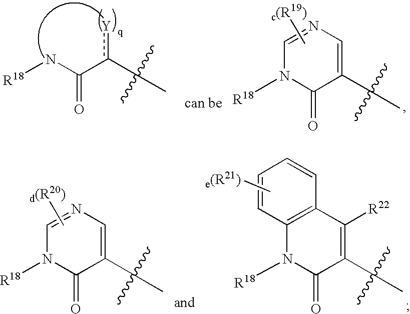

and pharmaceutical acceptable salts thereof
wherein R¹⁹, R²⁰, R²¹ and R²⁸ at each occurrence are independently selected from the group consisting of halogen, alkyl, alkenyl, alkynyl, alkoxy, alkenoxy, alkynoxy, thioalkoxy, hydroxyalkyl, aliphatic acyl, —CF₃, —OH, —CO₂H, —SH, —CN, —NO₂, —NH₂, allynylamino, alkoxycarbonyl, heterocycloyl, carboxy, —N(C₁-C₃ alkyl)-C(O)(C₁-C₃ alkyl), —NHC(O)N(C₁-C₃ alkyl)C(O)NH(C₁-C₃ alkyl), —NHC(O)NH(C₁-C₆ alkyl), —NHSO₂(C₁-C₃ alkyl), —NHSO₂(aryl), alkoxyalkyl, alkylamino, alkenylamino, di(C₁-C₃)amino, —C(O)O—(C₁-C₃)alkyl, —C(O)NH—(C₁-C₃)alkyl, —C(O)N(C₁-C₃ alkyl)₂, —CH═NOH, —PO₃H₂, —OPO₃H₂, haloalkyl, alkoxyalkoxy, carboxaldehyde, carboxamide, cycloalkyl, cycloalkenyl, cycloalkynyl, cycloalkylalkyl, aryl, aroyl, aryloxy, arylamino, biaryl, thioaryl, diarylamino, heterocyclyl, alkylaryl, aralkenyl, aralkyl, alkylheterocyclyl, heterocyclylalkyl, sulfonyl, —SO₂—(C₁-C₃ alkyl), —SO₃—(C₁-C₃ alkyl), sulfonamido, carbamate, aryloxyalkyl and —C(O)NH(benzyl) groups;
R¹⁸ is selected from the group consisting of alkyl, alkenyl, alkynyl, hydroxyalkyl, aliphatic acyl, alkynylamino, alkoxycarbonyl, heterocycloyl, —CH═NOH, haloalkyl, alkoxyalkoxy, carboxaldehyde, carboxamide, cycloalkyl, cycloalkenyl, cycloalkynyl, cycloalkylalkyl, aryl, aroyl, aryloxy, arylamino, biaryl, thioaryl, diarylamino, heterocyclyl, alkylaryl, aralkenyl, aralkyl, alkylheterocyclyl, heterocyclylalkyl, carbamate, aryloxyalkyl, hydrogen and —C(O)NH(benzyl) groups;
R²² is selected from the group consisting of hydrogen, halogen, alkyl, alkenyl, alkynyl, alkoxy, alkenoxy, alkynoxy, thioalkoxy, hydroxyalkyl, aliphatic acyl, —CF₃, —CO₂H, —SH, —CN, —NO₂, —NH₂, —OH, alkynylamino, alkoxycarbonyl, heterocycloyl, carboxy, —N(C₁-C₃ alkyl)-C(O)(C₁-C₃ alkyl), —NHC(O)N(C₁-C₃ alkyl)C(O)NH(C₁-C₃ alkyl), —NHC(O)NH(C₁-C₆ alkyl), —NHSO₂(C₁-C₃ alkyl), —NHSO2(aryl), alkoxyalkyl, alkylamino, alkenylamino, di(C₁-C₃)amino, —C(O)O—(C₁-C_{b 3})alkyl, —C(O)NH—(C₁-C₃)alkyl, —C(O)N(C₁-C₃ alkyl)₂, —CH═NOH, —PO₃H₂, —OPO₃H₂, haloalkyl, alkoxyalkoxy, carboxaldehyde, carboxamide, cycloalkyl, cycloalkenyl, cycloalkynyl, cycloalkylalkyl, aryl, aroyl, aryloxy, arylamino, biaryl, thioaryl, diarylamino, heterocyclyl, alkylaryl, aralkenyl, aralkyl, alkylheterocyclyl, heterocyclylalkyl, sulfonyl, —SO₂—(C₁-C₃ alkyl), —SO₃—(C₁-C₃ alkyl), sulfonamido, carbamate, aryloxyalkyl and —C(O)NH(benzyl) groups;
c is an integer of zero to two;
d is an integer of zero to three;
e is an integer of zero to four; and
i is an integer of zero to two.
   In one embodiment, R¹⁸ is aralkyl; R⁴ is aryl; T is (CH₂)_{b} where b is zero; L is (CH₂)ₙ where n is zero; and, B, R⁶, R⁷, R⁹ and R¹⁰ are each independently hydrogen.

Even more specifically, the compsitions of this invention comprise compounds of Formula IV 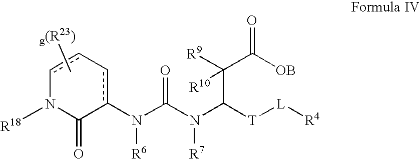
wherein T is selected from the group consisting of C(O) and (CH₂)_{b} wherein b is an integer of from 0 to 3;
L is selected from the group consisting of O, NR¹¹, S, and (CH₂)ₙ wherein n is an integer of 0 or 1;
g is an integer of from 0 to 7;
B is selected from the group consisting of hydrogen, alkyl, alkenyl, alkynyl, hydroxyalkyl, haloalkyl, —CF3, cycloalkyl, cycloalkenyl, cycloalkynyl, cycloalkylalkyl, aryl, heterocyclyl, alkylaryl, aralkenyl, aralkyl, alkylheterocyclyl, heterocyclylalkyl and aryloxyalkyl; and
R⁴, R⁹, R¹⁰ and R²³ at each occurrence are independently selected from the group consisting of hydrogen, halogen, alkyl, alkenyl, alkynyl, alkoxy, alkenoxy, alkynoxy, thioalkoxy, hydroxyalkyl, aliphatic acyl, —CF₃, —CO₂H, —SH, —CN, —NO₂, —NH₂, —OH alkynylamino, alkoxycarbonyl, heterocycloyl, carboxy, —N(C₁-C₃ alkyl)-C(O)(C₁-C₃alkyl), —NHC(O)N(C₁-C₃ alkyl)C(O)NH(C₁-C₃ alkyl)-NHC(O)NH(C₁-C₆alkyl), —NHSO₂(C₁-C₃ alkyl), —NHSO₂(aryl), alkoxyalkyl, alkylamino, alkenylamino, di(C₁-C₃)amino, —C(O)O—(C₁-C₃)alkyl, —C(O)NH—(C₁-C₃)alkyl, —C(O)N(C₁-C₃ alkyl)₂, —CH═NOH, —PO₃H₂, —OPO₃H₂, haloalkyl, alkoxyalkoxy, carboxaldehyde, carboxamide, cycloalkyl, cycloalkenyl, cycloalkynyl, cycloalkylalkyl, aryl, aroyl, aryloxy, arylamino, biaryl, thioaryl, diarylamino, heterocyclyl, alkylaryl, aralkenyl, aralkyl, alkylheterocyclyl, heterocyclylalkyl, sulfonyl, —SO₂—(C₁-C₃ alkyl), —SO₃—(C₁-C₃ alkyl), sulfonamido, carbamate, aryloxyalkyl and —C(O)NH(benzyl) groups; and
R⁶, R⁷, R¹¹ and R¹⁸ are each independently selected from the group consisting of alkyl, alkenyl, alkynyl, hydroxyalkyl, aliphatic acyl, alkynylamino, alkoxycarbonyl, heterocycloyl, —CH═NOH, haloalkyl, alkoxyalkoxy, carboxaldehyde, carboxamide, cycloalkyl, cycloalkenyl, cycloalkynyl, cycloalkylalkyl, aryl, aroyl, aryloxy, arylamino, biaryl, thioaryl, diarylamino, heterocyclyl, alkylaryl, aralkenyl, aralkyl, alkylheterocyclyl, heterocyclylalkyl, carbamate, aryloxyalkyl, hydrogen and —C(O)NH(benzyl) groups;
wherein B, R⁴, R⁶, R⁷, R⁹, R¹⁰, R¹¹, R¹⁸ and R²³ are unsubstituted or substituted with at least one electron donating or electron withdrawing group;
   wherein when L is NR¹¹, R⁴ and R¹¹ taken together may form a ring; and
   wherein R⁹ and R¹⁰ taken together may form a ring;

or a pharmaceutically acceptable salt thereof and one or more other therapeutic compounds.

Presently preferred compositions of the present invention comprise compounds of Formula V 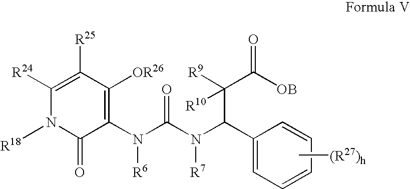
wherein h is an integer of zero to five;
B is selected from the group consisting of hydrogen, alkyl, alkenyl, alkynyl, hydroxyalkyl, haloalkyl, —CF₃, cycloalkyl, cycloalkenyl, cycloalkynyl, cycloalkylalkyl, aryl, heterocyclyl, alkylaryl, aralkenyl, aralkyl, alkylheterocyclyl, heterocyclylalkyl and aryloxyalkyl;
R⁹, R¹⁰, R²⁴, and R²⁵ are each independently selected from the group consisting of hydrogen, halogen, alkyl, alkenyl, alkynyl, alkoxy, alkenoxy, alkynoxy, thioalkoxy, hydroxyalkyl, aliphatic acyl, —CF₃, —CO₂H, —SH, —CN, —NO₂, —NH₂, —OH, alkynylamino, alkoxycarbonyl, heterocycloyl, carboxy, —N(C₁-C₃ alkyl)-C(O)(C₁-C₃ alkyl), —NHC(O)N(C₁-C₃ alkyl)C(O)NH(C₁-C₃alkyl), —NHC(O)NH(C₁-C₆ alkyl), —NHSO₂(C₁-C₃ alkyl), —NHSO₂(aryl), alkoxyalkyl, alkylamino, alkenylamino, di(C₁-C₃)amino, —C(O)O—(C₁-C₃)alkyl, —C(O)NH—(C₁-C₃)alkyl, —C(O)N(C₁-C₃ alkyl)₂, —CH═NOH, —PO₃H₂, —OPO₃H₂, haloalkyl, alkoxyalkoxy, carboxaldehyde, carboxamide, cycloalkyl, cycloalkenyl, cycloalkynyl, cycloalkylalkyl, aryl, aroyl, aryloxy, arylamino, biaryl, thioaryl, diarylamino, heterocyclyl, alkylaryl, aralkenyl, aralkyl, alkylheterocyclyl, heterocyclylalkyl, sulfonyl, —SO₂—(C₁-C₃ alkyl), —SO₃—(C₁-C₃ alkyl), sulfonamido, carbamate, aryloxyalkyl and —C(O)NH(benzyl) groups;
R²⁷, at each occurrence, is independently selected from the group consisting of halogen, alkyl, alkenyl, alkynyl, alkoxy, alkenoxy, alkynoxy, thioalkoxy, hydroxyalkyl, aliphatic acyl, —CF₃, —CO₂H, —SH, —CN, —NO₂, —NH₂, —OH, alkynylamino, alkoxycarbonyl, heterocycloyl, carboxy, —N(C₁-C₃ alkyl)-C(O)(C₁-C₃ alkyl), —NHC(O)N(C₁-C₃ alkyl)C(O)NH(C₁-C₃ alkyl), —NHC(O)NH(C₁-C₆ alkyl), —NHSO₂(C₁-C₃ alkyl), —NHSO₂(aryl), —N(C₁-C₃ alkyl)SO₂(C₁-C₃alkyl), —N(C₁-C₃ alkyl)SO₂(aryl), —C alkoxyalkyl,alkylamino, alkenylamino, di(C₁-C₃)amino, —C(O)O—(C₁-C₃)alkyl, —C(O)NH—(C₁-C₃)alkyl, —C(O)N(C₁-C₃ alkyl)₂, —CH═NOH, —PO₃H₂, —OPO₃H₂, haloalkyl, alkoxyalkoxy, carboxaldehyde, carboxamide, cycloalkyl, cycloalkenyl, cycloalkynyl, cycloalkylalkyl, aryl, aroyl, aryloxy, arylamino, biaryl, thioaryl, diarylamino, heterocyclyl, alkylaryl, aralkenyl, aralkyl, alkylheterocyclyl, heterocyclylalkyl, sulfonyl, —SO₂—(C₁-C₃ alkyl), —SO₃—(C₁-C₃ alkyl), sulfonamido, carbamate, aryloxyalkyl and —C(O)NH(benzyl) groups;
R⁶, R⁷ and R¹⁸ are each independently selected from the group consisting of alkyl, alkenyl, alkynyl, hydroxyalkyl, aliphatic acyl, alkynylamino, alkoxycarbonyl, heterocycloyl, —CH═NOH, haloalkyl, alkoxyalkoxy, carboxaldehyde, carboxamide, cycloalkyl, cycloalkenyl, cycloalkynyl, cycloalkylalkyl, aryl, aroyl, aryloxy, arylamino, biaryl, thioaryl, diarylamino, heterocyclyl, alkylaryl, aralkenyl, aralkyl, alkylheterocyclyl, heterocyclylalkyl, carbamate, aryloxyalkyl, hydrogen and —C(O)NH(benzyl) groups; and,
R²⁶ is selected from the group consisting of hydrogen, alkyl, alkenyl, alkynyl, hydroxyalkyl, aliphatic acyl, —CF₃, alkoxycarbonyl, heterocycloyl, carboxy, —C(O)O—(C₁-C₃)alkyl, —C(O)NH—(C₁-C₃)alkyl, —C(O)N(C₁-C₃ alkyl)₂, —PO₃H₂, haloalkyl, carboxamide, cycloalkyl, cycloalkenyl, cycloalkynyl, cycloalkylalkyl, aryl, aroyl, biaryl, heterocyclyl, alkylaryl, aralkenyl, aralkyl, alkylheterocyclyl, heterocyclylalkyl, sulfonyl, —SO₂—(C₁-C₃ alkyl), sulfonamido, aryloxyalkyl and —C(O)NH(benzyl) groups;
wherein B, R⁶, R⁷, R⁹, R¹⁰, R¹⁸, R²⁴, R²⁵, R²⁶and R²⁷ are unsubstituted or substituted with at least one electron donating or electron withdrawing group;
   wherein R¹⁸ and R²⁴ taken together may form a ring;
   R²⁴ and R²⁵ taken together may form a ring;
   R²⁵ and R²⁶ taken together may form a ring;
   and wherein R⁹ and R¹⁰ taken together may form a ring;

or a pharmaceutically acceptable salt thereof and one or more other therapeutic compounds.

Presently preferred compounds of Formula V have B, R⁶, R⁷, R⁹, R¹⁰, R²⁴, R²⁵ and R²⁶ each independently hydrogen and R¹⁸ as substituted or unsubstituted aralkyl.

Other presently preferred compositions of the present invention comprise compounds of Formula VI 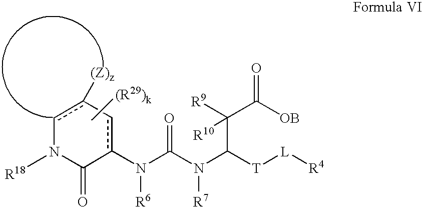
wherein Z, at each occurrence, is independently selected from the group consisting of C(O), N, CR³⁰, C(R³¹)(R³²), NR³³ CH, O and S;
z is an integer of from 3 to 6;
k is an integer of from 0 to 5;
T is selected from the group consisting of C(O) and (CH₂)_{b} wherein b is an integer of from 0 to 3;
L is selected from the group consisting of O, NR¹¹, S, and (CH₂)ₙ wherein n is an integer of 0 or 1;
R⁶, R⁷, R¹¹, R¹⁸ and R³³ are each independently selected from the group consisting of alkyl, alkenyl, alkynyl, hydroxyalkyl, aliphatic acyl, allynylamino, alkoxycarbonyl, heterocycloyl, —CH═NOH, haloalkyl, alkoxyalkoxy, carboxaldehyde, carboxamide, cycloalkyl, cycloalkenyl, cycloalkynyl, cycloalkylalkyl, aryl, aroyl, aryloxy, arylamino, biaryl, thioaryl, diarylamino, heterocyclyl, alkylaryl, aralkenyl, aralkyl, alkylheterocyclyl, heterocyclylalkyl, carbamate, aryloxyalkyl, hydrogen and —C(O)NH(benzyl) groups;
B is selected from the group consisting of hydrogen, alkyl, alkenyl, alkynyl, hydroxyalkyl, haloalky, —CF₃, cycloalkyl, cycloalkenyl, cycloalkynyl, cycloalkylakyl, aryl, heterocyclyl, alkylanyl, aralkenyl, aralkyl, alkylheterocyckyl, heterocyclylalkyl and aryoxyalkyl;
R⁴, R⁹, R¹⁰, R³⁰, R³¹ and R³² at each occurrence are independently selected from the group consisting of hydrogen, halogen, alkyl, alkenyl, alkynyl, alkoxy, alkenoxy, alkynoxy, thioalkoxy, hydroxyalkyl, aliphatic acyl, —CF₃, —CO₂H, —SH, —CN, —NO₂, —NH₂, —OH, alkynylamino, alkoxycarbonyl, heterocycloyl, carboxy, —N(C₁-C₃ alkyl)-C(O)(C₁-C₃ alkyl), —NHC(O)N(C₁-C₃ alkyl)C(O)NH(C₁-C₃alkyl), —NHC(O)NH(C₁-C₆ alkyl), —NHSO₂(C₁-C₃ alkyl), —NHSO₂(aryl), alkoxyalkyl, alkylamino, alkenylamino, di(C₁-C₃)amino, —C(O)O—(C₁-C₃)alkyl, —C(O)NH—(C₁-C₃)alkyl, —C(O)N(C₁-C₃ alkyl)₂, —CH═NOH, —PO₃H₂, —OPO₃H₂, haloalkyl, alkoxyalkoxy, carboxaldehyde, carboxamide, cycloalkyl, cycloalkenyl, cycloalkynyl, cycloalkylalkyl, aryl, aroyl, aryloxy, arylamino, biaryl, thioaryl, diarylamino, heterocyclyl, alkylaryl, aralkenyl, aralkyl, alkylheterocyclyl, heterocyclylalkyl, sulfonyl, —SO₂—(C₁-C₃ alkyl), —SO₃—(C₁-C₃ alkyl), sulfonamido, carbamate, aryloxyalkyl and —C(O)NH(benzyl) groups; and,
R²⁹, at each occurrence, is independently selected from the group consisting of halogen, alkyl, alkenyl, alkynyl, alkoxy, alkenoxy, alkynoxy, thioalkoxy, hydroxyalkyl, aliphatic acyl, —CF₃, —CO₂H, —SH, —CN, —NO₂, —NH₂, —OH, alkynylamino, alkoxycarbonyl, heterocycloyl, carboxy, —N(C₁-C₃ alkyl)-C(O)(C₁-C₃ alkyl), —NHC(O)N(C₁-C₃ alkyl)C(O)NH(C₁-C₃ alkyl), —NHC(O)NH(C₁-C₆alkyl), —NHSO₂(C₁-C₃ alkyl), —NHSO₂(aryl), alkoxyalkyl, alkylamino, alkenylamino, di(C₁-C₃)amino, —C(O)O—(C₁-C₃)alkyl, —C(O)NH—(C₁-C₃)alkyl, —C(O)N(C₁-C₃ alkyl)₂, —CH═NOH, —PO₃H₂, —OPO₃H₂, haloalkyl, alkoxyalkoxy, carboxaldehyde, carboxamide, cycloalkyl, cycloalkenyl, cycloalkynyl, cycloalkylalkyl, aryl, aroyl, aryloxy, arylamino, biaryl, thioaryl, diarylamino, heterocyclyl, alkylaryl, aralkenyl, aralkyl, alkylheterocyclyl, heterocyclylalkyl, sulfonyl, —SO₂—(C₁-C₃ alkyl), —SO_{3—(C}₁-C₃ alkyl), sulfonamido, carbamate, aryloxyalkyl and —C(O)NH(benzyl) groups;
wherein B, R⁴, R⁶, R⁷, R⁹, R¹⁰, R¹¹, R¹⁸, R²⁹, R³⁰, R³¹, R³² and R³³ are unsubstituted or substituted with at least one electron donating or electron withdrawing group;
   wherein when L is NR¹¹, R⁴ and R¹¹ taken together may form a ring; and
   wherein R⁹ and R¹⁰ taken together may form a ring;

or a pharmaceutically acceptable salt thereof and one or more other therapeutic compounds.

Presently preferred compounds that inhibit α₄β1 binding and are useful in the compositions of the present invention include (3S)-3-[({[1-(2-chlorobenzyl)-4-hydroxy-5-methyl-2-oxo-1,2-dihydropyridin-3-yl]amino}carbonyl)amino]-3-(4-methylphenyl)propanoic acid and pharmaceutically acceptable salts thereof; (3S)-3-[({[1-(2-chlorobenzyl)-4-hydroxy-2-oxo-2,5,6,7-tetrahydro-1H-cyclopenta[b]pyridin-3-yl]amino}carbonyl)amino]-3-(4-methylphenyl)propanoic acid and pharmaceutically acceptable salts thereof; and (3S)-3-[({[1-(2-chlorobenzyl)4-hydroxy-5-methyl-2-oxo-1,2-dihydropyridin-3-yl]amino}carbonyl)amino]-3-[3-(diethylamino)phenyl]propanoic acid and pharmaceutically acceptable salts thereof.

The pharmaceutical compositions of the present invention also comprise other therapeutic compounds and physiologically acceptable diluents.

The invention also provides a method for treating disease states mediated by α₄β₁ binding which comprises administration of an effective amount of a composition of the present invention to an afflicted patient.

The present invention is also directed to a method for treating disease states mediated by α₄β₁ binding which comprises co-administering a therapeutically effective amount of a combination of a compound of Formulae I, II, III, IV, V or VI and a therapeutically effective amount of one or more other therapeutic compounds to an afflicted patient.

The present invention is further directed to a kit comprising in a single package, one container comprising a compound that inhibits binding of α₄β₁ integrin to its receptors in a pharmaceutically acceptable carrier and one or more separate containers comprising other therapeutic compounds in pharmaceutically acceptable carriers, with the compound that inhibits binding of α₄β₁ integrin to its receptors and the other therapeutic compounds being present in amounts such that the combination is effective to treat disease states mediated by α₄β₁ integrin binding.

### DETAILED DESCRIPTION OF THE INVENTION

### Definitions of Terms

The term “alkyl” as used herein, alone or in combination, refers to C₁-C₁₂ straight or branched, substituted or unsubstituted saturated chain radicals derived from saturated hydrocarbons by the removal of one hydrogen atom, unless the term alkyl is preceded by a Cₓ-C_{y} designation. Representative examples of alkyl groups include methyl, ethyl, n-propyl, iso-propyl, n-butyl, sec-butyl, iso-butyl, and tert-butyl among others.

The term “alkenyl” as used herein, alone or in combination, refers to a substituted or unsubstituted straight-chain or substituted or unsubstituted branched-chain alkenyl radical containing from 2 to 10 carbon atoms. Examples of such radicals include, but are not limited to, ethenyl, E- and Z-pentenyl, decenyl and the like.

The term “alkynyl” as used herein, alone or in combination, refers to a substituted or unsubstituted straight or substituted or unsubstituted branched chain alkynyl radical containing from 2 to 10 carbon atoms. Examples of such radicals include, but are not limited to ethynyl, propynyl, propargyl, butynyl, hexynyl, decynyl and the like.

The term “lower” modifying “alkyl”, “alkenyl”, “alkynyl” or “alkoxy” refers to a C₁-C₆ unit for a particular functionality. For example lower alkyl means C₁-C₆ alkyl.

The term “aliphatic acyl” as used herein, alone or in combination, refers to radicals of formula alkyl-C(O)—, alkenyl-C(O)— and alkynyl-C(O)— derived from an alkane-, alkene- or alkyncarboxylic acid, wherein the terms “alkyl”, “alkenyl” and “alkynyl” are as defined above. Examples of such aliphatic acyl radicals include, but are not limited to, acetyl, propionyl, butyryl, valeryl, 4-methylvaleryl, acryloyl, crotyl, propiolyl and methylpropiolyl, among others.

The term “cycloalkyl” as used herein refers to an aliphatic ring system having 3 to 10 carbon atoms and 1 to 3 rings, including, but not limited to cyclopropyl, cyclopentyl, cyclohexyl, norbornyl, and adamantyl among others. Cycloalkyl groups can be unsubstituted or substituted with one, two or three substituents independently selected from lower alkyl, haloalkyl, alkoxy, thioalkoxy, amino, alkylamino, dialkylamino, hydroxy, halo, mercapto, nitro, carboxaldehyde, carboxy, alkoxycarbonyl and carboxamide.

“Cycloalkyl” includes cis or trans forms. Furthermore, the substituents may either be in endo or exo positions in the bridged bicyclic systems.

The term “cycloalkenyl” as used herein alone or in combination refers to a cyclic carbocycle containing from 4 to 8 carbon atoms and one or more double bonds. Examples of such cycloalkenyl radicals include, but are not limited to, cyclopentenyl, cyclohexenyl, cyclopentadienyl and the like.

The term “cycloalkylalkyl” as used herein refers to a cycloalkyl group appended to a lower alkyl radical, including, but not limited to cyclohexylmethyl.

The term “halo” or “halogen” as used herein refers to I, Br, Cl or F.

The term “haloalkyl” as used herein refers to a lower alkyl radical, to which is appended at least one halogen substituent, for example chloromethyl, fluoroethyl, trifluoromethyl and pentafluoroethyl among others.

The term “alkoxy” as used herein, alone or in combination, refers to an alkyl ether radical, wherein the term “alkyl” is as defined above. Examples of suitable alkyl ether radicals include, but are not limited to, methoxy, ethoxy, n-propoxy, iso-propoxy, n-butoxy, iso-butoxy, sec-butoxy, tert-butoxy and the like.

The term “alkoxyalkyl” as used herein, refers to R_{y}—O—R_{z}, wherein R_{y} is lower alkyl as defined above, and R_{z} is alkylene (—(CH₂)_{w}—) wherein w is an integer of from one to six. Representative examples include methoxymethyl, methoxyethyl, and ethoxyethyl among others.

The term “alkenoxy” as used herein, alone or in combination, refers to a radical of formula alkenyl-O, provided that the radical is not an enol ether, wherein the term “alkenyl” is as defined above. Examples of suitable alkenoxy radicals include, but are not limited to, allyloxy, E- and Z- 3-methyl-2-propenoxy and the like.

The term “alkynoxy” as used herein, alone or in combination, refers to a radical of formula alkynyl-O, provided that the radical is not an -ynol ether. Examples of suitable alkynoxy radicals include, but are not limited to, propargyloxy, 2-butynyloxy and the like.

The term “carboxy” as used herein refers to —C(O)O—.

The term “thioalkoxy” refers to a thioether radical of formula alkyl-S—, wherein “alkyl” is as defined above.

The term “sulfonamido” as used herein refers to —SO₂NH₂.

The term “carboxaldehyde” as used herein refers to —C(O)R wherein R is hydrogen.

The terms “carboxamide” or “amide” as used herein refer to —C(O)NRₐR_{b} wherein Rₐ and R_{b} are each independently hydrogen, alkyl or any other suitable substituent.

The term “alkoxyalkoxy” as used herein refers to R_{c}O—R_{d}O— wherein R_{c} is lower alkyl as defined above and R_{d} is alkylene wherein alkylene is —(CH₂)ₙ— wherein n¹ is an integer from 1 to 6. Representative examples of alkoxyalkoxy groups include methoxymethoxy, ethoxymethoxy, t-butoxymethoxy among others.

The term “alkylamino” as used herein refers to R_{c}NH— wherein Rₑ is a lower alkyl group, for example, ethylamino, butylamino, among others.

The term “alkenylamino” as used herein, alone or in combination, refers to a radical of formula alkenyl-NH— or (alkenyl)₂N—, wherein the term “alkenyl” is as defined above, provided that the radical is not an enamine. An example of such alkenylamino radical is the alkylamino radical.

The term “alkynylamino” as used herein, alone or in combination, refers to a radical of formula alkynyl-NH— or (alkynyl)₂N— wherein the term “alkynyl” is as defined above, provided that the radical is not an amine. An example of such alkynylamino radicals is the propargyl amino radical.

The term “dialkylamino” as used herein refers to R_{f}R_{g}N— wherein R_{f} and R_{g} are independently selected from lower alkyl, for example diethylamino, and methyl propylamino, among others.

The term “alkoxycarbonyl” as used herein refers to an alkoxyl group as previously defined appended to the parent molecular moiety through a carbonyl group. Examples of alkoxycarbonyl include methoxycarbonyl, ethoxycarbonyl, and isopropoxycarbonyl among others.

The term “aryl” or “aromatic” as used herein alone or in combination refers to a substituted or unsubstituted carbocyclic aromatic group having about 6 to 12 carbon atoms such as phenyl, naphthyl, indenyl, indanyl, azulenyl, fluorenyl and anthracenyl; or a heterocyclic aromatic group containing at least one endocyclic N, O or S atom such as furyl, thienyl, pyridyl, pyrrolyl, oxazolyl, thiazolyl, imidazolyl, pyrazolyl, 2-pyrazolinyl, pyrazolidinyl, isoxazolyl, isothiazolyl, 1,2,3-oxadiazolyl, 1,2,3-triazolyl, 1,3,4-thiadiazolyl, pyridazinyl, pyrimidinyl, pyrazinyl, 1,3,5-triazinyl, 1,3,5-trithianyl, indolizinyl, indolyl, isoindolyl, 3H-indolyl, indolinyl, benzo[b]furanyl, 2,3-dihydrobenzofuranyl, benzo[b]thiophenyl, 1H-indazolyl, benzimidazolyl, benzthiazolyl, purinyl, 4H-quinolizinyl, isoquinolinyl, cinnolinyl, phthalazinyl, quinazolinyl, quinoxalinyl, 1,8-naphthridinyl, pteridinyl, carbazolyl, acridinyl, phenazinyl, phenothiazinyl, phenoxyazinyl, pyrazolo[1,5-c]triazinyl and the like. “Aralkyl” and “alkylaryl” employ the term “alkyl” as defined above. Rings may be multiply substituted.

The term “aralkyl” as used herein, alone or in combination, refers to an aryl substituted alkyl radical, wherein the terms “alkyl” and “aryl” are as defined above. Examples of suitable aralkyl radicals include, but are not limited to, phenylmethyl, phenethyl, phenylhexyl, diphenylmethyl, pyridylmethyl, tetrazolyl methyl, furylmethyl, imidazolyl methyl, indolylmethyl, thienylpropyl and the like.

The term “aralkenyl” as used herein, alone or in combination, refers to an aryl substituted alkenyl radical, wherein the terms “aryl” and “alkenyl” are as defined above.

The term “arylamino” as used herein, alone or in combination, refers to a radical of formula aryl-NH—, wherein “aryl” is as defined above. Examples of arylamino radicals include, but are not limited to, phenylamino(anilido), naphthlamino, 2-, 3-, and 4-pyridylamino and the like.

The term “benzyl” as used herein refers to C₆H₅—CH₂—.

The term “biaryl” as used herein, alone or in combination, refers to a radical of formula aryl-aryl, wherein the term “aryl” is as defined above.

The term “thioaryl” as used herein, alone or in combination, refers to a radical of formula aryl-S—, wherein the term “aryl” is as defined above. An example of a thioaryl radical is the thiophenyl radical.

The term “aroyl” as used herein, alone or in combination, refers to a radical of formula aryl-CO—, wherein the term “aryl” is as defined above. Examples of suitable aromatic acyl radicals include, but are not limited to, benzoyl, 4-halobenzoyl, 4-carboxybenzoyl, naphthoyl, pyridylcarbonyl and the like.

The term “heterocyclyl” as used herein, alone or in combination, refers to a non-aromatic 3- to 10-membered ring containing at least one endocyclic N, O, or S atom. The heterocycle may be optionally aryl-fused. The heterocycle may also optionally be substituted with at least one substituent which is independently selected from the group consisting of hydrogen, halogen, hydroxyl, amino, nitro, trifluoromethyl, trifluoromethoxy, alkyl, aralkyl, alkenyl, alkynyl, aryl, cyano, carboxy, carboalkoxy, carboxyalkyl, oxo, arylsulfonyl and aralkylaminocarbonyl among others.

The term “alkylheterocyclyl” as used herein refers to an alkyl group as previously defined appended to the parent molecular moiety through a heterocyclyl group, including but not limited to 2-methyl-5-thiazolyl, 2-methyl-1-pyrrolyl and 5-ethyl-2-thienyl.

The term “heterocyclylalkyl” as used herein refers to a heterocyclyl group as previously defined appended to the parent molecular moiety through an alkyl group, including but not limited to 2-thienylmethyl, 2-pyridinylmethyl and 2-(1-piperidinyl)ethyl.

The term “heterocycloyl” as used herein refers to radicals of the formula heterocyclyl-C(O)—, wherein the term “hetercyclyl” is as defined above.

The term “aminal” as used herein refers to a hemi-acetal of the structure RₕC(NRᵢRⱼ)(NRₖRₗ)— wherein Rₕ, Rᵢ, Rⱼ, Rₖ and Rₗ are each independently hydrogen, alkyl or any other suitable substituent.

The term “ester” as used herein refers to —C(O)Rₘ, wherein Rₘ is hydrogen, alkyl or any other suitable substituent.

The term “carbamate” as used herein refers to compounds based on carbamic acid NH₂C(O)OH.

The term “optical isomers” as used herein refers to compounds which differ only in the stereochemistry of at least one atom, including enantiomers, diastereomers and racemates.

Use of the above terms is meant to encompass substituted and unsubstituted moieties. Substitution may be by one or more groups such as alcohols, ethers, esters, amides, sulfones, sulfides, hydroxyl, nitro, cyano, carboxy, amines, heteroatoms, lower alkyl, lower alkoxy, lower alkoxycarbonyl, alkoxyalkoxy, acyloxy, halogens, trifluoromethoxy, trifluoromethyl, alkyl, aralkyl, alkenyl, alkynyl, aryl, cyano, carboxy, carboalkoxy, carboxyalkyl, cycloalkyl, cycloalkylalkyl, heterocyclyl, alkylheterocyclyl, heterocyclylalkyl, oxo, arylsulfonyl and aralkylaminocarbonyl or any of the substituents of the preceding paragraphs or any of those substituents either attached directly or by suitable linkers. The linkers are typically short chains of 1-3 atoms containing any combination of —C—, —C(O)—, —NH—, —S—, —S(O)—, —O—, —C(O)O— or —S(O)O—. Rings may be substituted multiple times.

The terms “electron-withdrawing” or “electron-donating” refer to the ability of a substituent to withdraw or donate electrons relative to that of hydrogen if hydrogen occupied the same position in the molecule. These terms are well-understood by one skilled in the art and are discussed in Advanced Organic Chemistry by J. March, 1985, pp. 16-18, incorporated herein by reference. Electron withdrawing groups include halo, nitro, carboxyl, lower alkenyl, lower alkynyl, carboxaldehyde, carboxyamido, aryl, quaternary ammonium, trifluoromethyl, sulfonyl and aryl lower alkanoyl among others. Electron donating groups include such groups as hydroxy, lower alkyl, amino, lower alkylamino, di(lower alkyl)amino, aryloxy, mercapto, lower alkylthio, lower alkylmercapto, and disulfide among others. One skilled in the art will appreciate that the aforesaid substituents may have electron donating or electron withdrawing properties under different chemical conditions. Moreover, the present invention contemplates any combination of substituents selected from the above-identified groups.

The most preferred electron donating or electron withdrawing substituents are halo, nitro, alkanoyl, carboxaldehyde, arylalkanoyl, aryloxy, carboxyl, carboxamide, cyano, sulfonyl, sulfoxide, heterocyclyl, guanidine, quaternary ammonium, lower alkenyl, lower alkynyl, sulfonium salts, hydroxy, lower alkoxy, lower alkyl, amino, lower alkylamino, di(lower alkyl)amino, amine lower alkyl mercapto, mercaptoalkyl, alkylthio, carboxy lower alkyl, arylalkoxy, alkanoylamino, alkanoy(lower alkyl)amino, lower alkylsufonylamino, arylsulfonylamino, alkylsulfonyl(lower alkyl)amino, arylsulfonyl(lower alkyl)amino, lower alkylcarboxamide, di(lower alkyl)carboxamide, sulfonamide, lower alkylsulfonamide, di(lower alkyl)sulfonamide, lower alkylsulfonyl, arylsulfonyl and alkyldithio.

As used herein, the term “composition” is intended to encompass a product comprising the specified ingredients in the specified amounts, as well as any product which results, directly or indirectly, from a combination of the specified ingredients in the specified amounts.

As used herein, the term “mammals” includes humans and other animals.

The ring defined by Y in Formulae I, II and III can be a mono-cyclic heterocycle or aromatic ring, or can be a bicyclic ring.

The dotted lines used in Formulae I, II, III, IV and VI indicate that the bond at that location can be either single or double. The bond between the atoms Y and W for example can be a single or double bond if Y and/or W is a substitutent such as N, C or CH. Therefore, the ring defined by Y in the Formulae can be either saturated or unsaturated, depending upon which W and/or Y is selected. In Formulae IV and VI, the dotted line indicates that the nitrogen-containing ring optionally contains double bonds at the indicated locations.

In the Formulae, certain R groups potentially substitute their associated rings a number of times. R¹⁹, R²⁰, R²¹, R²³, R²⁷ , R²⁸ , R²⁹ and R²⁵ may each substitute their associated rings more than once. For example for R¹⁹, when c is zero, the associated ring is unsubstituted, having hydrogens at the C-2 and C4 positions; and for R²³, when g is zero, hydrogens are at the C-2-C-5 positions.

Suitable substituents for the aryl, alkyl, cycloalkyl, heterocyclyl groups or the ring defined by Y and W in the formulae described above, when present, include alcohols, amines, heteroatoms, or any combination of aryl, alkoxy, alkoxyalkoxy, alkyl, cycloalkyl or heterocyclyl groups either attached directly, or via suitable linkers. The linkers are typically short chains of 1-3 atoms containing any combination of C, C═O, CO₂, O, N, S, S═O, SO₂, as for example ethers, amides, amines, ureas, sulfamides, sulfonamides, among others. For example, R¹, R², R³, R⁵, R⁶, R⁷ and R⁸ in the above formulae may independently be, but are not limited to: hydrogen, alkyl, phenyl, thienylmethyl, isobutyl, n-butyl, 2-thienylmethyl, 1,3-thiazol-2-yl-methyl, benzyl, thienyl, 3-pyridinylmethyl, 3-methyl-1-benzothiophen-2-yl, allyl, 3-methoxybenzyl, propyl, 2-ethoxyethyl, cyclopropylmethyl, benzylsulfanylmethyl, benzylsulfonylmethyl, phenylsulfanylmethyl, phenethylsulfanylmethyl, 3-phenylpropylsulfanylmethyl, 4-((2-toluidinocarbonyl)amino)benzyl, 2-pyridinylethyl, 2-(1H-indol-3-yl)ethyl, 1H-benzimidazol-2-yl, 4-piperidinylmethyl, 3-hydroxy4-methoxybenzyl, 4-hydroxyphenethyl, 4-aminobenzyl, phenylsulfonylmethyl, 4-(acetylamino)phenyl, 4-methoxyphenyl, 4-aminophenyl, 4-chlorophenyl, (4-(benzylsulfonyl)amino)phenyl, (4-(methylsulfonyl)amino)phenyl, 2-aminophenyl, 2-methylphenyl, isopropyl, 2-oxo-1-pyrrolidinyl, 3-(methylsulfanyl)propyl, (propylsulfanyl)methyl, octylsulfanylmethyl, 3-aminophenyl, 4-((2-toluidinocarbonyl)amino)phenyl, 2-((methylbenzyl)amino)benzyl, methylsulfanylethyl, hydroxy, chloro, fluoro, bromo, ureido, amino, methanesulfonylamino, acetylamino, ethylsulfanylmethyl, 2-chlorobenzyl, 2-bromobenzyl, 2-fluorobenzyl, 2-chloro-6-fluorobenzyl, 2-chloro-4-fluorobenzyl, 2,4-dichlorobenzyl, 2-chloro-6-methoxybenzyl, 2-cyanobenzyl, 2,6-difluorobenzyl, 2-chloro-5-(trifluoromethyl)benzyl, 2-chloro-6-methylbenzyl, 2,6-dimethoxybenzyl, 2-chloro-5-(methylsulfonyl)benzyl, 2-chloro-6-cyanobenzyl, 2-chloro-6-ethoxybenzyl, 2-chloro-5-methoxybenzyl, 2-chloro-5-fluorobenzyl, 5-chloro-2-fluorobenzyl, ethyl, propyl, butyl, pentyl, cyclopropyl, tert-butylamino, propylamino, 4methyl-1-piperazinyl, 1-azetidinyl, 4-morpholino, (4-carboxyphenyl)amino, pivaloylamino, ((tert-butylamino) carbonyl)amino, trifluoromethyl, benzyloxy, 2-(2-methoxyethoxy)ethoxy, 2-(2-(2-methoxyethoxy)ethoxy)ethoxy and 2-(2-(2-(2-ethoxyethoxy)ethoxy)ethoxy)ethoxy.

The R⁴ substituent for the formulae above may be, but is not limited to 1,3-benzodioxol-5-yl, 1-naphthyl, thienyl, 4-isobutoxyphenyl, 2,6-dimethylphenyl, allyloxyphenyl, 3-bromo4-methoxyphenyl, 4-butoxyphenyl, 1-benzofuran-2-yl, 2-thienylmethyl, phenyl, methylsulfanyl, phenylsulfanyl, phenethylsulfanyl, 4-bromo-2-thienyl, 3-methyl-2-thienyl, 4-methylphenyl, 3,5-bis(methyloxy)phenyl, 4-(methyloxy)phenyl, 4-fluorophenyl, 3-(methyloxy)phenyl, 3,4,5-tris(methyloxy)phenyl, 2,3-dihydro-1-benzofuran-5-yl, 3-fluorophenyl, 4-(trifluoromethyl)phenyl, 4-fluoro-3-(trifluoromethyl)phenyl, 4-(1,1-dimethylethyl)phenyl, 3,5-dimethylphenyl, 4-hydroxyphenyl, 3,4-dimethylphenyl, 3-methyl-4-(methyloxy)phenyl, 4hydroxy-3-methylphenyl, 3-methylphenyl, 2,3-dihydro-inden-5-yl, 2-methylphenyl, 2,6-bis(methyloxy)phenyl, 2,6-dihydroxyphenyl, 4-chlorophenyl, 3-chlorophenyl, 3,4-dichlorophenyl, 4-(trifluoromethyl)oxy)phenyl, 4-ethylphenyl, 4-(ethyloxy)phenyl, methyl, 2-propyl, 4,5-dihydro-1,3-oxazol-2-yl, 3-(trifluoromethyl)phenyl, 4-(trifluoromethoxy)phenyl, 2,3-dihydro-1,4-benzodioxin-6-yl, 7-methoxy-1,3-benzodioxol-5-yl, 3-ethoxy-4-methoxyphenyl, 3,4-dimethoxyphenyl, 3,4-diethoxyphenyl, 3-ethoxyphenyl, 3-methoxy-4-methylphenyl, 3,5-dimethoxy4-methylphenyl, 3-propoxyphenyl, 3-butoxyphenyl, 3-2-methoxyethoxy)phenyl, 3,4-dipropoxyphenyl, 3-(difluoromethoxy)phenyl, 2-naphthyl, 3-isopropoxyphenyl, 1-methyl-1H-indol-5-yl, 2,3-dihydro-1-benzofuran-5-yl, 1,3-diethyl-2-oxo-2,3-dihydro-1H-benzimidazol-5-yl, 3-(trifluoromethoxy)phenyl, 1-methyl-1H-indol-6-yl, 3-(cyclopropoxy)phenyl, 3-(cyclopropylmethoxy)phenyl, 3-(difluoromethoxy)phenyl, 3-(1,1,2,2-tetrafluoroethoxy)phenyl, 1-ethyl-1H-indol-5-yl, 3-diethylamino)phenyl, 6-methoxy-2-naphthyl, 3-[(methylsulfonyl)amino]phenyl, 3-[methyl(methylsulfonyl)amino]phenyl, 3-[ethyl(methylsulfonyl)amino]phenyl, 1H-indol-5-yl, 3-fluoro4-methoxyphenyl and 3-(difluoromethyl)phenyl.

Two independent R¹, R², R³ or R⁵ groups taken together may be linked to form a ring.

R⁴ and R¹¹ may be linked to form a ring such as 1-pyrrolidino, 1-piperidino, 4-methyl-1-piperazino, 4-acetyl-1-piperazino and 4-morpholino among others.

R⁹ and R¹⁰ may be linked to form a ring such as cyclopropyl, cyclobutyl, cyclopentyl, and cyclohexyl among others.

Representative disease targets of the compositions of the present invention include inflammatory diseases such as psoriasis, asthma, atheroscloerosis, autoimmune diseases such as multiple sclerosis and Guillan-Barr Syndrome, rheumatoid arthritis, transplant and graft v. host disease, inflammatory bowel disease, chronic obstructive pulmonary disease and reperfusion injury.

One or more α₄β₁ binding (VLA-4) inhibitors may be combined with one or more other therapeutic compounds. The other therapeutic compounds include compounds for treating an inflammatory disease, i.e., inflammatory response, as, for example, IL-5 antagonists for growth, maturation and survival of eosinophils; CCR-3 antagonists for chemotaxis of eosinophils; corticosteroids for general suppression of inflammation; antihistamines for histamine early phase blockages; Leukotriene antagonists for LTD bronchoconstrictor and LTB eosinophil chemotaxis; COX-I and COX-II inhibitors for prostaglandin production; mast cell stabilizers such as Chromolyn; anti-IL-5 or anti-IgE; IL-5 synthesis and release inhibitors; selectin antagonists, CD20 antagonists for suppression of B-cell mediated inflammation and syk tyrosine kinase inhibitors.

Agents known in the treatment of rheumatoid arthritis, transplant and graft v. host disease, inflammatory bowel disease, chronic obstructive pulmonary disease and multiple sclerosis which can be administered in combination with the α₄β₁ binding inhibitors of the present invention are as follows:

solid organ transplant rejection and graft v. host disease: immune suppressants such as cyclosporine rapamycin and Interleukin-10 (IL-10), tacrolimus, antilymphocyte globulin, OKT-3 antibody, and steroids;

inflammatory bowel disease: IL-10 (see U.S. Pat. No. 5,368,854), steroids and azulfidine;

rheumatoid arthritis: methotrexate, azathioprine, cyclophosphamide, steroids and mycophenolate mofetil;

multiple sclerosis: interferon-beta, interferon-alpha, and steroids.

Non limitative examples of antihistamines include: astemizole, azatadine, azelastine, acrivastine, brompheniramine, certirizine, chlorpheniramine, clemastine, cyclizine, carebastine, cyproheptadine, carbinoxamine, descarboethoxyloratadine (also known as SCH-34117), doxylamine, dimethindene, ebastine, epinastine, efletirizine, fexofenadine, hydroxyzine, ketotifen, loratadine, levocabastine, mizolastine, equitazine, mianserin, noberastine, meclizine, norastermizole, picumast, pyrilamine, promethazine, terfenadine, tripelennamine, temelastine, trimeprazine and triprolidine.

The term “leukotriene antagonist” includes any agent or compound that inhibits, restrains, retards or otherwise interacts with the action or activity of leukotrienes. Non-limitative examples of leukotriene inhibitors include montelukast [R-(E)]-1[[[1-[3-[2-(7-chloro-2-quinolinyl)-ethenyl] phenyl]-3[2-(1-hydroxy-1-methylethyl)phenyl]propyl]thio]methyl]cyclo-propaneacetic acid and its sodium salt, described in EP 0 480 717; 1-(((R)-(3-(2-(6,7-difluoro-2-quinolinyl)ethenyl)phenyl)-3-(2-(2-hydroxy-2-propyl)phenyl)thio)methylcyclopropaneacetic acid, and its sodium salt, described in WO 97/28797 and U.S. Pat. No. 5,270,324; 1-(((1(R)-3(3-(2-(2,3-dichlorothieno[3,2-b]pyridin-5-yl)-(E)-ethenyl)phenyl)-3-(2-( -hydroxy-1-methylethyl)phenyl)propyl)thio)methyl)cyclopropaneacetic acid, and its sodium salt, described in WO 97/28797 and U.S. Pat. No. 5,472,964; pranlukast, N-[4-oxo-2-(H-tetrazol-5-yl)-4H-1-benzopyran-8-yl]-p-(4-phenylbutoxy)benzamide) described in WO 97/28797 and EP 173,516; zafirlukast, (cyclopentyl-3-[2-methoxy-4-[(o-tolylsulfonyl)carbamoyl]benzyl]-1-methylindole-5-carbamate) described in WO 97/28797 and EP 199,543; and [2-[[2(4-tert-butyl-2-thiazolyl)-5-benzofuranyl]oxymethyl]phenyl]acetic acid, described in U.S. Pat. No. 5,296,495 and Japanese patent JP08325265A.

Other therapeutic agents that may be used in conjunction with the compounds that inhibit binding of α₄β₁ integrin to its receptors include:

β-agonists: albuterol, salmeterol, formoterol, levabuterol, terbutaline, pirbuterol, metaprotrenol

muscarininc antagonists: ipratropium bromide, tiatropium bromide

PDE 4 inhibitors: roflumilast, theophylline, rolipram, piclamilast, cilomilast, CDP-840

immunosuppressives: methotrexate, leflunomide, sulfasalazine, cyclosporin

steroids: prednisolone, fluticasone, triamcinolone, beclomethasone, mometasone, budisainide, betamethasone, dexamethasone, prednisone, flunisolide, cortisone

COX-I inhibitors: aspirin, piroxicam

COX-II inhibitors: rofecoxib, celecoxib, valdecoxib, etoricoxib

TNF-α inhibitor: infliximab, etanercept

Anti-IgE antibody: omalizumab

p38 MAP kinase inhibitor: VX-745, BIRB-796

tryptase inhubitor: BMS-262084

anticytokine/antichemokine: RO-3202947, UCB-35625

vaccine: Allervax Cat

cromolyn: oral cromolyn formulation from Emisphere Technologies

Anti-CD20 antibody: rituximab

syk tyrosine kinase inhibitor: R-112

Actual dosage levels of active ingredients in the pharmaceutical compositions of this invention can be varied so as to obtain an amount of the active compounds which are effective to achieve the desired therapeutic response for a particular patient, compositions and mode of administration. The selected dosage level will depend upon the activity of the particular compounds, the route of administration, the severity of the condition being treated and the condition and prior medical history of the patient being treated. However, it is within the skill of the art to start doses of the compound at levels lower than required to achieve the desired therapeutic effect and to gradually increase the dosage until the desired effect is achieved.

The phrase “therapeutically effective amount” of the composition of the invention means a sufficient amount of the active compounds to treat disorders, at a reasonable benefit/risk ratio applicable to any medical treatment. It will be understood, however, that the total daily usage of the compounds and compositions of the present invention will be decided by the attending physician within the scope of sound medical judgment. The specific therapeutically effective dose level for any particular patient will depend upon a variety of factors including the disorder being treated and the severity of the disorder, activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed; and like factors well known in the medical arts. For example, it is well within the skill of the art to start doses of the compound at levels lower than required to achieve the desired therapeutic effect and to gradually increase the dosage until the desired effect is achieved.

The total daily dose of the compositions of this invention administered to a human or lower animal may range from about 0.0001 to about 1000 mg/kg/day. For purposes of oral administration, more preferable doses can be in the range of from about 0.001 to about 5 mg/kg/day. If desired, the effective daily dose can be divided into multiple doses for purposes of administration; consequently, single dose compositions may contain such amounts or submultiples thereof to make up the daily dose.

The pharmaceutical compositions of this invention can be administered to humans and other mammals orally, rectally, parenterally, intracisternally, intravaginally, intraperitoneally, bucally or as an oral or nasal spray. The term “parenterally,” as used herein, refers to modes of administration which include intravenous, intramuscular, intraperitoneal, intrastemal, subcutaneous and intraarticular injection and infusion.

The present invention includes one or more compounds as described above formulated into compositions together with one or more non-toxic physiologically tolerable or acceptable diluents, carriers, adjuvants or vehicles that are collectively referred to herein as diluents, for parenteral injection, for intranasal delivery, for oral administration in solid or liquid form, for rectal or topical administration, among others.

The compositions can also be delivered through a catheter for local delivery at a target site, via an intracoronary stent (a tubular device composed of a fine wire mesh), or via a biodegradable polymer. The compounds may also be complexed to ligands, such as antibodies, for targeted delivery.

Compositions suitable for parenteral injection may comprise physiologically acceptable, sterile aqueous or nonaqueous solutions, dispersions, suspensions or emulsions and sterile powders for reconstitution into sterile injectable solutions or dispersions. Examples of suitable aqueous and nonaqueous carriers, diluents, solvents or vehicles include water, ethanol, polyols (propyleneglycol, polyethyleneglycol, glycerol, and the like), vegetable oils (such as olive oil), injectable organic esters such as ethyl oleate, and suitable mixtures thereof.

These compositions can also contain adjuvants such as preserving, wetting, emulsifying, and dispensing agents. Prevention of the action of microorganisms can be ensured by various antibacterial and antifungal agents, for example, parabens, chlorobutanol, phenol, sorbic acid, and the like. It may also be desirable to include isotonic agents, for example sugars, sodium chloride and the like. Prolonged absorption of the injectable pharmaceutical form can be brought about by the use of agents delaying absorption, for example, aluminum monostearate and gelatin.

Suspensions, in addition to the active compounds, may contain suspending agents, as for example, ethoxylated isostearyl alcohols, polyoxyethylene sorbitol and sorbitan esters, microcrystalline cellulose, aluminum metahydroxide, bentonite, agar-agar and tragacanth, or mixtures of these substances, and the like.

In some cases, in order to prolong the effect of the drug, it is desirable to slow the absorption of the drug from subcutaneous or intramuscular injection. This can be accomplished by the use of a liquid suspension of crystalline or amorphous material with poor water solubility. The rate of absorption of the drug then depends upon its rate of dissolution which, in turn, may depend upon crystal size and crystalline form. Alternatively, delayed absorption of a parenterally administered drug form is accomplished by dissolving or suspending the drug in an oil vehicle.

Injectable depot forms are made by forming microencapsule matrices of the drug in biodegradable polymers such as polylactide-polyglycolide. Depending upon the ratio of drug to polymer and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissues.

The injectable formulations can be sterilized, for example, by filtration through a bacterial-retaining filter or by incorporating sterilizing agents in the form of sterile solid compositions which can be dissolved or dispersed in sterile water or other sterile injectable medium just prior to use.

Solid dosage forms for oral administration include capsules, tablets, pills, powders and granules. In such solid dosage forms, the active compound may be mixed with at least one inert, pharmaceutically acceptable excipient or carrier, such as sodium citrate or dicalcium phosphate and/or a) fillers or extenders such as starches, lactose, sucrose, glucose, mannitol and silicic acid; b) binders such as carboxymethylcellulose, alginates, gelatin, polyvinylpyrrolidone, sucrose and acacia; c) humectants such as glycerol; d) disintegrating agents such as agar-agar, calcium carbonate, potato or tapioca starch, alginic acid, certain silicates and sodium carbonate; e) solution retarding agents such as paraffin; f) absorption accelerators such as quaternary ammonium compounds; g) wetting agents such as cetyl alcohol and glycerol monostearate; h) absorbents such as kaolin and bentonite clay and i) lubricants such as talc, calcium stearate, magnesium stearate, solid polyethylene glycols, sodium lauryl sulfate and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like.

The solid dosage forms of tablets, dragees, capsules, pills and granules can be prepared with coatings and shells such as enteric coatings and other coatings well-known in the pharmaceutical formulating art. They may optionally contain opacifying agents and may also be of a composition such that they release the active ingredient(s) only, or preferentially, in a certain part of the intestinal tract, optionally, in a delayed manner. Examples of embedding compositions which can be used include polymeric substances and waxes.

The active compounds can also be in micro-encapsulated form, if appropriate, with one or more of the above-mentioned excipients.

Liquid dosage forms for oral administration include pharmaceutically acceptable emulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethyl formamide, oils (in particular, cottonseed, groundnut, corn, germ, olive, castor and sesame oils), glycerol, tetrahydrofurfuryl alcohol, polyethylene glycols and fatty acid esters of sorbitan and mixtures thereof.

Besides inert diluents, the oral compositions may also include adjuvants such as wetting agents, emulsifying and suspending agents, sweetening, flavoring and perfuming agents.

Compositions for rectal or vaginal administration are preferably suppositories which can be prepared by mixing the compounds of this invention with suitable non-irritating excipients or carriers such as cocoa butter, polyethylene glycol or a suppository wax which are solid at room temperature but liquid at body temperature and therefore melt in the rectum or vaginal cavity and release the active compound.

Compounds useful in the compositions of the present invention can also be administered in the form of liposomes. As is known in the art, liposomes are generally derived from phospholipids or other lipid substances. Liposomes are formed by mono- or multi-lamellar hydrated liquid crystals which are dispersed in an aqueous medium. Any non-toxic, physiologically acceptable and metabolizable lipid capable of forming liposomes can be used. The present compositions in liposome form can contain, in addition to a compound of the present invention, stabilizers, preservatives, excipients and the like. The preferred lipids are natural and synthetic phospholipids and phosphatidyl cholines (lecithins) used separately or together.

Methods to form liposomes are known in the art. See, for example, Prescott, Ed., Methods in Cell Biology, Volume XIV, Academic Press, New York, N.Y. (1976), p. 33 et seq.

The term “pharmaceutically acceptable prodrugs” as used herein represents those prodrugs of the compounds of the present invention which are, within the scope of sound medical judgment, suitable for use in contact with the tissues of humans and lower animals without undue toxicity, irritation, allergic response, and the like, commensurate with a reasonable benefit/risk ratio, and effective for their intended use, as well as the zwitterionic forms, where possible, of the compounds of the invention. Prodrugs of the present invention may be rapidly transformed in vivo to the parent compound of the above formula, for example, by hydrolysis in blood. A thorough discussion is provided in T. Higuchi and V. Stella, Pro-drugs as Novel Delivery Systems. V. 14 of the A.C.S. Symposium Series, and in Edward B. Roche, ed., Bioreversible Carriers in Drug Design, American Pharmaceutical Association and Pergamon Press (1987), hereby incorporated by reference.

Compounds that are useful in the compositions of the present invention that are formed by in vivo conversion of a different compound that was administered to a mammal are intended to be included within the scope of the present invention.

Compounds that are useful in the compositions of the present invention may exist as stereoisomers wherein asymmetric or chiral centers are present. These stereoisomers are “R” or “S” depending on the configuration of substituents around the chiral carbon atom. The present invention contemplates various stereoisomers and mixtures thereof. Stereoisomers include enantiomers and diastereomers, and mixtures of enantiomers or diastereomers. Individual stereoisomers of compounds may be prepared synthetically from commercially available starting materials which contain asymmetric or chiral centers or by preparation of racemic mixtures followed by resolution well-known to those of ordinary skill in the art. These methods of resolution are exemplified by (1) attachment of a mixture of enantiomers to a chiral auxiliary, separation of the resulting mixture of diastereomers by recrystallization or chromatography and liberation of the optically pure product from the auxiliary or (2) direct separation of the mixture of optical enantiomers on chiral chromatographic columns.

Compounds useful in the compositions of the invention can exist in unsolvated as well as solvated forms, including hydrated forms, such as hemi-hydrates. In general, the solvated forms, with pharmaceutically acceptable solvents such as water and ethanol among others are equivalent to the unsolvated forms for the purposes of the invention.

A procedure in which a 26-amino acid peptide containing the CS 1 sequence of fibronectin with an N-terminal Cys (CDELPQLVTLPHPNLHGPEELDVPST) may be coupled to maleimide activated ovalbumin was used to determine the efficacy of the compounds synthesized. Bovine serum albumin (BSA) and CS 1 conjugated ovalbumin may be coated onto 96-well polystyrene plates at 0.5 ptg/ml in TBS (50 mM TRIS, pH 7.5; 150 mM NaCl) at 4′ C. for 16 hours. The plates may be washed three times with TBS and blocked with TBS containing 3% BSA at room temperature for 4 hours. Blocked plates may be marked three times in binding buffer (TBS; 1 MM MgC12; 1 mM CaC12; 1 mM MnC12) prior to assay. Ramos cells fluorescently labeled with calcein AM may be resuspended in binding buffer (107 cells/ml) and diluted 1:2 with same buffer with or without compound. 100 pM of compound should be added. The cells should be added immediately to the wells (2.5×105 cells/ well) and incubated for 30 minutes at 37 C. Following three washes with binding buffer, adherent cells should be lysed and quantitated using a fluorometer. IC50 is defined as the dose required to give 50% inhibition. The lower the IC50 value and the greater the percentage of inhibition, the more efficient the compound is at prevention of cell adhesion.

1. A pharmaceutical composition comprising a compound of the structure 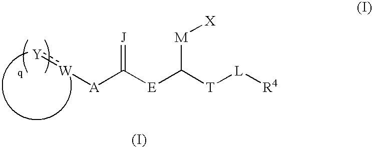
wherein Y, at each occurrence, is independently selected from the group consisting of C(O), N, CR¹, C(R²)(R³), NR⁵, CH, O and S;
q is an integer of from 3 to 10;
A is selected from the group consisting of O, S, C(R¹⁶ )(R¹⁷) and NR⁶;
E is selected from the group consisting of CH₂, O, S, and NR⁷;
J is selected from the group consisting of O, S and NR⁸;
T is selected from the group consisting of C(O) and (CH₂)_{b} wherein b is an integer of from 0 to 3;
M is selected from the group consisting of C(R⁹)(R¹⁰) and (CH₂)ᵤ, wherein u is an integer of from 0 to 3;
L is selected from the group consisting of O, NR¹¹, S, and (CH₂)ₙ wherein n is an integer of 0 or 1;
X is selected from the group consisting of CO₂B, PO₃H₂, SO₃H, SO₂NH₂, SO₂NHCOR¹², OPO₃H₂, C(O)NHC(O)R¹³, C(O)NHSO₂R¹⁴, hydroxyl, tetrazolyl and hydrogen;
W is selected from the group consisting of C, CR¹⁵ and N;
B is selected from the group consisting of hydrogen, alkyl, alkenyl, alkynyl, hydroxyalkyl, haloalkyl, —CF₃, cycloalkyl, cycloalkenyl, cycloalkynyl, cycloalkylalkyl, aryl, heterocyclyl, alkylaryl, aralkenyl, aralkyl, alkylheterocyclyl, heterocyclylalkyl and aryloxyalkyl; and
R¹, R², R³, R⁴, R⁵, R⁶, R⁷, R⁸, R⁹, R¹⁰, R¹¹, R¹², R¹³ , R¹⁴, R¹⁵, R¹⁶ and R¹⁷ at each occurrence are independently selected from the group consisting of hydrogen, halogen, alkyl, alkenyl, alkynyl, alkoxy, alkenoxy, alkynoxy, thioalkoxy, hydroxyalkyl, aliphatic acyl, —CF₃, —CO₂H, —SH, —CN, —NO₂, —NH₂, —OH, alkynylamino, alkoxycarbonyl, heterocycloyl, carboxy, —N(C₁-C₃ alkyl)-C(O)(C₁-C₃ alkyl), —NHC(O)N(C₁-C₃ alkyl)C(O)NH(C₁-C₃alkyl), —NHC(O)NH(C₁-C₆ alkyl), —NHSO₂(C₁-C₃ alkyl), —NHSO₂(aryl), alkoxyalkyl, alkylamino, alkenylamino, di(C₁-C₃)amino, —C(O)O—(C₁-C₃)alkyl, —C(O)NH—(C₁-C₃)alkyl, —C(O)N(C₁-C₃ alkyl)₂, —CH═NOH, —PO₃H₂, —OPO₃H₂, haloalkyl, alkoxyalkoxy, carboxaldehyde, carboxamide, cycloalkyl, cycloalkenyl, cycloalkynyl, cycloalkylalkyl, aryl, aroyl, aryloxy, arylamino, biaryl, thioaryl, diarylamino, heterocyclyl, alkylaryl, aralkenyl, aralkyl, alkylheterocyclyl, heterocyclylalkyl, sulfonyl, —SO₂—(C₁-C₃ alkyl), —SO₃—(C₁-C₃ alkyl), sulfonamido, carbamate, aryloxyalkyl and —C(O)NH(benzyl) groups;
wherein B, R¹,R², R³, R⁴, R⁵, R⁶, R⁷, R⁸, R⁹, R¹⁰, R¹¹, R¹², R¹³, R¹⁴, R¹⁵, R¹⁶ and R¹⁷ are unsubstituted or substituted with at least one electron donating or electron withdrawing group;
wherein when L is NR¹¹, R⁴ and R¹¹ taken together may form a ring;
and wherein when M is C(R⁹)(R¹⁰), R⁹ and R¹⁰ taken together may form a ring;
and wherein when A is NR⁶ and at least one Y is CR¹, R¹ and R⁶ taken together may form a ring;
or a pharmaceutically acceptable salt thereof;
one or more other therapeutically active compounds and a pharmacologically acceptable diluent.

2. A composition of claim 1 wherein
A is NR⁶;
E is NR⁷;
J is O;
M is C(R⁹)(R¹⁰);
q is 4 or 5;
T is (CH₂)_{b} wherein b is 0;
L is (CH₂)ₙ wherein n is 0;
X is CO₂B;
W is C or CR¹⁵;
R⁴ is selected from the group consisting of aryl, alkylaryl, aralkyl, heterocyclyl, alkylheterocyclyl and heterocyclylalkyl; and
R⁶, R⁷, R⁹, R¹⁰ and R¹⁵ are independently selected from the group consisting of hydrogen and lower alkyl.

3. A pharmaceutical composition comprising a compound of the structure 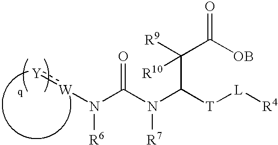
wherein Y, at each occurrence, is independently selected from the group consisting of C(O), N, CR¹, C(R²)(R³), NR⁵, CH, O and S;
q is an integer of from 3 to 7;
T is selected from the group consisting of C(O) and (CH₂)_{b} wherein b is an integer of 0 to 3;
L is selected from the group consisting of O, NR¹¹, S, and (CH₂)ₙ wherein n is an integer of 0 or 1;
W is selected from the group consisting of C, CR¹⁵ and N;
B is selected from the group consisting of hydrogen, alkyl, alkenyl, alkynyl, hydroxyalkyl, haloalkyl, —CF₃, cycloalkyl, cycloalkenyl, cycloalkynyl, cycloalkylalkyl, aryl, heterocyclyl, alkylaryl, aralkenyl, aralkyl, alkylheterocyclyl, heterocyclylalkyl and aryloxyalkyl; and
R¹, R², R³, R⁴, R⁵, R⁶, R⁷, R⁹, R¹⁰, R¹¹ and R¹⁵ are independently selected from the group consisting of hydrogen, halogen, alkyl, alkenyl, alkynyl, alkoxy, alkenoxy, alkynoxy, thioalkoxy, hydroxyalkyl, aliphatic acyl, —CF₃, —CO₂H, —SH, —CN, —NO₂, —NH₂, —OH, alkynylamino, alkoxycarbonyl, heterocycloyl, carboxy, —N(C₁-C₃ alkyl)-C(O)(C₁-C₃ alkyl), —NHC(O)N(C₁-C₃ alkyl)C(O)NH(C₁-C₃alkyl), —NHC(O)NH(C₁-C₆ alkyl), —NHSO₂(C₁-C₃ alkyl), —NHSO₂(aryl), alkoxyalkyl, alkylamino, alkenylamino, di(C₁-C₃)amino, —C(O)O—(C₁-C₃)alkyl, —C(O)NH—(C₁-C₃)alkyl, —C(O)N(C₁-C₃ alkyl)₂, —CH═NOH, —PO₃H₂, —OPO₃H₂, haloalkyl, alkoxyalkoxy, carboxaldehyde, carboxamide, cycloalkyl, cycloalkenyl, cycloalkynyl, cycloalkylalkyl, aryl, aroyl, aryloxy, arylamino, biaryl, thioaryl, diarylamino, heterocyclyl, alkylaryl, aralkenyl, aralkyl, alkylheterocyclyl, heterocyclylalkyl, sulfonyl, —SO₂—(C₁-C₃ alkyl), —SO₃—(C₁-C₃ alkyl), sulfonamido, carbamate, aryloxyalkyl and —C(O)NH(benzyl) groups;
wherein B, R¹, R², R³, R⁴, R⁵, R⁶, R⁷, R⁹, R¹⁰, R¹¹ and R¹⁵ are unsubstituted or substituted with at least one electron donating or electron withdrawing group;
wherein when L is NR¹¹, R⁴ and R¹¹ taken together may form a ring;
and wherein R⁹ and R¹⁰ taken together may form a ring;
and wherein when at least one Y is CR¹, R¹ and R⁶ taken together may form a ring;
or a pharmaceutically acceptable salt thereof;
one or more other therapeutically active compounds and a pharmacologically acceptable diluent.

4. A composition of claim 3 wherein
q is 4 or 5;
W is C or CR¹⁵;
T is (CH₂)_{b} wherein b is 0;
L is (CH₂)ₙ wherein n is 0;
R⁴ is selected from the group consisting of aryl, alkylaryl, aralkyl, heterocyclyl, alkylheterocyclyl and heterocyclylalkyl; and
R⁶, R⁷, R⁹, R¹⁰ and R¹⁵ are independently selected from the group consisting of hydrogen and lower alkyl

5. A pharmaceutical composition comprising a compound of the structure 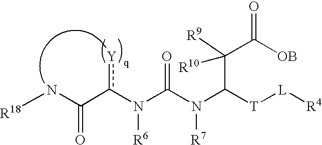
wherein Y, at each occurrence, is independently selected from the group consisting of C(O), N, CR¹, C(R²)(R³), NR⁵, CH, O and S;
q is an integer of from 2 to 5;
T is selected from the group consisting of C(O) and (CH₂)_{b} wherein b is an integer of 0 to 3;
L is selected from the group consisting of O, NR¹¹, S, and (CH₂)ₙ wherein n is an integer of 0 or 1;
R⁵, R⁶, R⁷, R¹¹ and R¹⁸ are each independently selected from the group consisting of alkyl, alkenyl, alkynyl, hydroxyalkyl, aliphatic acyl, alkynylamino, alkoxycarbonyl, heterocycloyl, —CH═NOH, haloalkyl, alkoxyalkoxy, carboxaldehyde, carboxamide, cycloalkyl, cycloalkenyl, cycloalkynyl, cycloalkylalkyl, aryl, aroyl, aryloxy, arylamino, biaryl, thioaryl, diarylamino, heterocyclyl, alkylaryl, aralkenyl, aralkyl, alkylheterocyclyl, heterocyclylalkyl, carbamate, aryloxyalkyl, hydrogen and —C(O)NH(benzyl) groups;
B is selected from the group consisting of hydrogen, alkyl, alkenyl, alkynyl, hydroxyalkyl, haloalkyl, —CF₃, cycloalkyl, cycloalkenyl, cycloalkynyl, cycloalkylalkyl, aryl, heterocyclyl, alkylaryl, aralkenyl, aralkyl, alkylheterocyclyl, heterocyclylalkyl and aryloxyalkyl; and
R¹, R², R³, R⁴, R⁹ and R¹⁰ are independently selected from the group consisting of hydrogen, halogen, halkyl, alkenyl, alkynyl, alkoxy, alkenoxy, alkynoxy, thioalkoxy, hydroxyalkyl, aliphatic acyl, —CF₃, —CO₂H, —SH, —CN, —NO₂, —NH₂, —OH, alkynylamino, alkoxycarbonyl, heterocycloyl, carboxy, —N(C₁-C₃ alkyl)-C(O)(C₁-C₃ alkyl), —NHC(O)N(C₁-C₃ alkyl)C(O)NH(C₁-C₃alkyl), —NHC(O)NH(C₁-C₆ alkyl), —NHSO₂(C₁-C₃ alkyl), —NHSO₂(aryl), alkoxyalkyl,alkylamino, alkenylamino, di(C₁-C₃)amino, —C(O)O—(C₁-C₃)alkyl, —C(O)NH—(C₁-C₃)alkyl, —C(O)N(C₁-C₃ alkyl)₂, —CH═NOH, —PO₃H₂, —OPO₃H₂, haloalkyl, alkoxyalkoxy, carboxaldehyde, carboxamide, cycloalkyl, cycloalkenyl, cycloalkynyl, cycloalkylalkyl, aryl, aroyl, aryloxy, arylamino, biaryl, thioaryl, diarylamino, heterocyclyl, alkylaryl, aralkenyl, aralkyl, alkylheterocyclyl, heterocyclylalkyl, sulfonyl, —SO₂—(C₁-C₃ alkyl), —SO₃—(C₁-C₃ alkyl), sulfonamido, carbamate, aryloxyalkyl and —C(O)NH(benzyl) groups;
wherein B, R¹, R² , R³, R⁴, R⁵, R⁶, R⁷, R⁹, R¹⁰, R¹¹ and R¹⁸ are unsubstituted or substituted with at least one electron donating or electron withdrawing group;
wherein when L is NR¹¹, R⁴ and R¹¹ taken together may form a ring;
and wherein R⁹ and R¹⁰ taken together may form a ring;
and wherein when at least one Y is CR¹, R¹ and R⁶ taken together may form a ring;
or a pharmaceutically acceptable salt thereof, one or more other therapeutically active compounds and a pharmacalogically acceptable diluent.

6. A composition of claim 5 wherein R¹⁸ is selected from the group consisting of
hydrogen, alkyl, aryl, aralkyl, cycloalkyl, alkylheterocyclyl, heterocyclylalkyl and heterocyclyl;
T is (CH₂)_{b} wherein b is 0;
L is (CH₂)ₙ wherein n is 0;
Y is selected from the group consisting of CR¹ and C(R²)(R³) and
q is 2 or 3.

7. A composition of claim 5 wherein 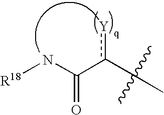
is selected from the group consisting of 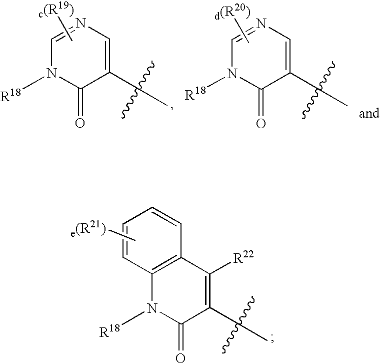
wherein R¹⁹, R²⁰ , R²¹ and R²⁸ at each occurrence are independently selected from the group consisting of halogen, alkyl, alkenyl, alkynyl, alkoxy, alkenoxy, alkynoxy, thioalkoxy, hydroxyalkyl, aliphatic acyl, —CF₃, —OH, —CO₂H, —SH, —CN, —NO₂, —NH₂, alkynylamino, alkoxycarbonyl, heterocycloyl, carboxy, —N(C₁-C₃ alkyl)-C(O)(C₁-C₃ alkyl), —NHC(O)N(C₁-C₃ alkyl)C(O)NH(C₁-C₃alkyl), —NHC(O)NH(C₁-C₆ alkyl), —NHSO₂(C₁-C₃ alkyl), —NHSO2(aryl), alkoxyalkyl, alkylamino, alkenylamino, di(C₁-C₃)amino, —C(O)O—(C₁-C₃)alkyl, —C(O)NH—(C₁-C₃)alkyl, —C(O)N(C₁-C₃ alkyl)₂, —CH═NOH, —PO₃H₂, —OPO₃H₂, haloalkyl, alkoxyalkoxy, carboxaldehyde, carboxamide, cycloalkyl, cycloalkenyl, cycloalkynyl, cycloalkylalkyl, aryl, aroyl, aryloxy, arylamino, biaryl, thioaryl, diarylamino, heterocyclyl, alkylaryl, aralkenyl, aralkyl, alkylheterocyclyl, heterocyclylalkyl, sulfonyl, —SO₂—(C₁-C₃ alkyl), —SO₃—(C₁-C₃ alkyl), sulfonamido, carbamate, aryloxyalkyl and —C(O)NH(benzyl) groups;
R¹⁸ is selected from the group consisting of alkyl, alkenyl, alkynyl, hydroxyalkyl, aliphatic acyl, alkynylamino, alkoxycarbonyl, heterocycloyl, —CH═NOH, haloalkyl, alkoxyalkoxy, carboxaldehyde, carboxamide, cycloalkyl, cycloalkenyl, cycloalkynyl, cycloalkylalkyl, aryl, aroyl, aryloxy, arylamino, biaryl, thioaryl, diarylamino, heterocyclyl, alkylaryl, aralkenyl, aralkyl, alkylheterocyclyl, heterocyclylalkyl, carbamate, aryloxyalkyl, hydrogen and —C(O)NH(benzyl) groups;
R²² is selected from the group consisting of hydrogen, halogen, alkyl, alkenyl, alkynyl, alkoxy, alkenoxy, alkynoxy, thioalkoxy, hydroxyalkyl, aliphatic acyl, —CF₃, —CO₂H, —SH, —CN, —NO₂, —NH₂, —OH, alkynylamino, alkoxycarbonyl, heterocycloyl, carboxy, —N(C₁-C₃ alkyl)-C(O)(C₁-C₃ alkyl), —NHC(O)N(C₁-C₃ alkyl)C(O)NH(C₁-C₃alkyl), —NHC(O)NH(C₁-C₆ alkyl), —NHSO₂(C₁-C₃ alkyl), —NHSO₂(aryl), alkoxyalkyl, alkylamino, alkenylamino, di(C₁-C₃)amino, —C(O)O—(C₁-C₃)alkyl, —C(O)NH—(C₁-C₃)alkyl, —C(O)N(C₁-C₃ alkyl)₂, —CH═NOH, —PO₃H₂, —OPO₃H₂, haloalkyl, alkoxyalkoxy, carboxaldehyde, carboxamide, cycloalkyl, cycloalkenyl, cycloalkynyl, cycloalkylalkyl, aryl, aroyl, aryloxy, arylamino, biaryl, thioaryl, diarylamino, heterocyclyl, alkylaryl, aralkenyl, aralkyl, alkylheterocyclyl, heterocyclylalkyl, sulfonyl, —SO₂—(C₁-C₃ alkyl), —SO₃—(C₁-C₃ alkyl), sulfonamido, carbamate, aryloxyalkyl and —C(O)NH(benzyl) groups;
c is an integer of zero to two;
d is an integer of zero to three;
e is an integer of zero to four; and
i is an integer of zero to two.

8. The composition of claim 5 wherein R¹⁸ is aralkyl;
R⁴ is aryl;
T is (CH₂)_{b} where b is zero;
L is (CH₂)ₙ where n is zero; and,
B, R⁶, R⁷, R⁹ and R¹⁰ are each independently hydrogen.

9. A pharmaceutical composition comprising a compound of the structure 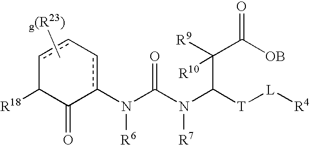
wherein T is selected from the group consisting of C(O) and (CH₂)_{b} wherein b is an integer of from 0 to 3;
L is selected from the group consisting of O, NR¹¹, S, and (CH₂)ₙ wherein n is an integer of 0 or 1;
g is an integer of from 0 to 7;
B is selected from the group consisting of hydrogen, alkyl, alkenyl, alkynyl, hydroxyalkyl, haloalkyl, —CF₃, cycloalkyl, cycloalkenyl, cycloalkynyl, cycloalkylalkyl, aryl, heterocyclyl, alkylaryl, aralkenyl, aralkyl, alkylheterocyclyl, heterocyclylalkyl and aryloxyalkyl; R⁴, R⁹, R¹⁰ and R²³ at each occurrence are independently selected from the group consisting of hydrogen, halogen, alkyl, alkenyl, alkynyl, alkoxy, alkenoxy, alkynoxy, thioalkoxy, hydroxyalkyl, aliphatic acyl, —CF₃, —CO₂H, —SH, —CN, —NO₂, —NH₂, —OH, alkynylamino, alkoxycarbonyl, heterocycloyl, carboxy, —N(C₁-C₃ alkyl)-C(O)(C₁-C₃ alkyl), —NHC(O)N(C₁-C₃ alkyl)C(O)NH(C₁-C₃alkyl), —NHC(O)NH(C₁-C₆ alkyl), —NHSO₂(C₁-C₃ alkyl), —NHSO₂(aryl), alkoxyalkyl,alkylamino, alkenylamino, di(C₁-C₃)amino, —C(O)O—(C₁-C₃)alkyl, —C(O)NH—(C₁-C₃)alkyl, —C(O)N(C₁-C₃ alkyl)₂, —CH═NOH, —PO₃H₂, —OPO₃H₂, haloalkyl, alkoxyalkoxy, carboxaldehyde, carboxamide, cycloalkyl, cycloalkenyl, cycloalkynyl, cycloalkylalkyl, aryl, aroyl, aryloxy, arylamino, biaryl, thioaryl, diarylamino, heterocyclyl, alkylaryl, aralkenyl, aralkyl, alkylheterocyclyl, heterocyclylalkyl, sulfonyl, —SO₂—(C₁-C₃ alkyl), —SO₃—(C₁-C₃ alkyl), sulfonamido, carbamate, aryloxyalkyl and —C(O)NH(benzyl) groups;
R⁶, R⁷, R¹¹ and R¹⁸ are each independently selected from the group consisting of alkyl, alkenyl, alkynyl, hydroxyalkyl, aliphatic acyl, alkynylamino, alkoxycarbonyl, heterocycloyl, —CH═NOH, haloalkyl, alkoxyalkoxy, carboxaldehyde, carboxamide, cycloalkyl, cycloalkenyl, cycloalkynyl, cycloalkylalkyl, aryl, aroyl, aryloxy, arylamino, biaryl, thioaryl, diarylamino, heterocyclyl, alkylaryl, aralkenyl, aralkyl, alkylheterocyclyl, heterocyclylalkyl, carbamate, aryloxyalkyl, hydrogen and —C(O)NH(benzyl) groups;
wherein B, R⁴, R⁶, R⁷, R⁹, R¹⁰, R¹¹, R¹⁸ and R²³ are unsubstituted or substituted with at least one electron donating or electron withdrawing group;
wherein when L is NR¹¹, R⁴ and R¹¹ taken together may form a ring;
and wherein R⁹ and R¹⁰ taken together may form a ring;
or a pharmaceutically acceptable salt thereof;
one or more other therapeutically active compounds and a pharmacologically acceptable diluent.

10. A pharmaceutical composition comprising a compound of the structure 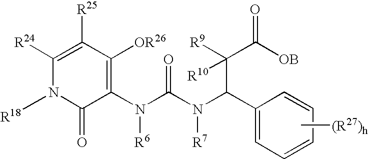
wherein h is an integer of zero to five;
B is selected from the group consisting of hydrogen, alkyl, alkenyl, alkynyl, hydroxyalkyl, haloalkyl, —CF₃, cycloalkyl, cycloalkenyl, cycloalkynyl, cycloalkylalkyl, aryl, heterocyclyl, alkylaryl, aralkenyl, aralkyl, alkylheterocyclyl, heterocyclylalkyl and aryloxyalkyl;
R⁹, R¹⁰, R²⁴, and R²⁵ are each independently selected from the group consisting of hydrogen, halogen, alkyl, alkenyl, alkynyl, alkoxy, alkenoxy, alkynoxy, thioalkoxy, hydroxyalkyl, aliphatic acyl, —CF₃, —CO₂H, —SH, —CN, —NO₂, —NH₂, —OH, alkynylamino, alkoxycarbonyl, heterocycloyl, carboxy, —N(C₁-C₃ alkyl)-C(O)(C₁-C₃ alkyl), —NHC(O)N(C₁-C₃ alkyl)C(O)NH(C₁-C₃alkyl), —NHC(O)NH(C₁-C₆ alkyl), —NHSO₂(C₁-C₃ alkyl), —NHSO₂(aryl), alkoxyalkyl, alkylamino, alkenylamino, di(C₁-C₃)amino, —C(O)O—(C₁-C₃)alkyl, —C(O)NH—(C₁-C₃)alkyl, —C(O)N(C₁-C₃ alkyl)₂, —CH═NOH, —PO₃H₂, —OPO₃H₂, haloalkyl, alkoxyalkoxy, carboxaldehyde, carboxamide, cycloalkyl, cycloalkenyl, cycloalkynyl, cycloalkylalkyl, aryl, aroyl, aryloxy, arylamino, biaryl, thioaryl, diarylamino, heterocyclyl, alkylaryl, aralkenyl, aralkyl, alkylheterocyclyl, heterocyclylalkyl, sulfonyl, —SO₂—(C₁-C₃ alkyl), —SO₃—(C₁-C₃ alkyl), sulfonamido, carbamate, aryloxyalkyl and —C(O)NH(benzyl) groups;
R²⁷, at each occurrence, is independently selected from the group consisting of halogen, hydroxyl, alkyl, alkenyl, alkynyl, alkoxy, alkenoxy, alkynoxy, thioalkoxy, hydroxyalkyl, aliphatic acyl, —CF₃, —CO₂H, —SH, —CN, —NO₂, —NH₂, alkynylamino, alkoxycarbonyl, heterocycloyl, carboxy, —N(C₁-C₃ alkyl)-C(O)(C₁-C₃ alkyl), —NHC(O)N(C₁-C₃ alkyl)C(O)NH(C₁-C₃alkyl), —NHC(O)NH(C₁-C₆ alkyl), —NHSO₂(C₁-C₃ alkyl), —NHSO₂(aryl), —N(C₁-C₃alkyl)SO₂(C₁-C₃alkyl), —N(C₁-C₃alkyl)SO₂(aryl), alkoxyalkyl, alkylamino, alkenylamino, di(C₁-C₃)amino, —C(O)O—(C₁-C₃)alkyl, —C(O)NH—(C₁-C₃)alkyl, —C(O)N(C₁-C₃ alkyl)₂, —CH═NOH, —PO₃H₂, —OPO₃H₂, haloalkyl, alkoxyalkoxy, carboxaldehyde, carboxamide, cycloalkyl, cycloalkenyl, cycloalkynyl, cycloalkylalkyl, aryl, aroyl, aryloxy, arylamino, biaryl, thioaryl, diarylamino, heterocyclyl, alkylaryl, aralkenyl, aralkyl, alkylheterocyclyl, heterocyclylalkyl, sulfonyl, —SO₂—(C₁-C₃ alkyl), —SO₃—(C₁-C₃ alkyl), sulfonamido, carbamate, aryloxyalkyl and —C(O)NH(benzyl) groups;
R⁶, R⁷ and R¹⁸ are each independently selected from the group consisting of alkyl, alkenyl, alkynyl, hydroxyalkyl, aliphatic acyl, alkynylamino, alkoxycarbonyl, heterocycloyl, —CH═NOH, haloalkyl, alkoxyalkoxy, carboxaldehyde, carboxamide, cycloalkyl, cycloalkenyl, cycloalkynyl, cycloalkylalkyl, aryl, aroyl, aryloxy, arylamino, biaryl, thioaryl, diarylamino, heterocyclyl, alkylaryl, aralkenyl, aralkyl, alkylheterocyclyl, heterocyclylalkyl, carbamate, aryloxyalkyl, hydrogen and —C(O)NH(benzyl) groups; and,
R26 is selected from the group consisting of hydrogen, alkyl, alkenyl, alkynyl, hydroxyalkyl, aliphatic acyl, —CF₃, alkoxycarbonyl, heterocycloyl, carboxy, —C(O)O—(C₁-C₃)alkyl, —C(O)NH—(C₁-C₃)alkyl, —C(O)N(C₁-C₃ alkyl)₂, —PO₃H₂, haloalkyl, carboxamide, cycloalkyl, cycloalkenyl, cycloalkynyl, cycloalkylalkyl, aryl, aroyl, biaryl, heterocyclyl, alkylaryl, aralkenyl, aralkyl, alkylheterocyclyl, heterocyclylalkyl, sulfonyl, —SO₂—(C₁-C₃ alkyl), sulfonamido, aryloxyalkyl and —C(O)NH(benzyl) groups;
wherein B, R⁶, R⁷, R⁹, R¹⁰, R₁₈, R²⁴, R²⁵, R²⁶ and R²⁷ are unsubstituted or substituted with at least one electron donating or electron withdrawing group;
wherein R¹⁸ and R²⁴ taken together may form a ring;
R²⁴ and R²⁵ taken together may form a ring;
R²⁵ and R²⁶ taken together may form a ring;
and wherein R⁹ and R¹⁰ taken together may form a ring;
or a pharmaceutically acceptable salt thereof;
one or more other therapeutically active compounds and a pharmacologically acceptable diluent.

11. The composition of claim 10 wherein B, R⁶, R⁷, R⁹, R¹⁰, R²⁴, R²⁵ and R²⁶ are each independently hydrogen and R¹⁸ is substituted or unsubstituted aralkyl.

12. A pharmaceutical composition comprising a compound of the structure 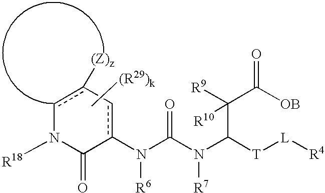
wherein Z, at each occurrence, is independently selected from the group consisting of C(O), N, CR³⁰, C(R³¹ )(R³²), NR³³, CH, O and S;
z is an integer of from 3 to 6;
k is an integer of from 0 to 5;
T is selected from the group consisting of C(O) and (CH₂)_{b} wherein b is an integer of from 0 to 3;
L is selected from the group consisting of O, NR¹¹, S, and (CH₂)ₙ wherein n is an integer of 0 or 1;
R⁶, R⁷, R¹¹, R¹⁸ and R³³ are each independently selected from the group consisting of alkyl, alkenyl, alkynyl, hydroxyalkyl, aliphatic acyl, alkynylamino, alkoxycarbonyl, heterocycloyl, —CH═NOH, haloalkyl, alkoxyalkoxy, carboxaldehyde, carboxamide, cycloalkyl, cycloalkenyl, cycloalkynyl, cycloalkylalkyl, aryl, aroyl, aryloxy, arylamino, biaryl, thioaryl, diarylamino, heterocyclyl, alkylaryl, aralkenyl, aralkyl, alkylheterocyclyl, heterocyclylalkyl, carbamate, aryloxyalkyl, hydrogen and —C(O)NH(benzyl) groups;
B is selected from the group consisting of hydrogen, alkyl, alkenyl, alkynyl, hydroxyalkyl, haloalkyl, —CF₃, cycloalkyl, cycloalkenyl, cycloalkynyl, cycloalkylalkyl, aryl, heterocyclyl, alkylaryl, aralkenyl, aralkyl, alkylheterocyclyl, heterocyclylalkyl and aryloxyalkyl;
R⁴, R⁹, R¹⁰, R³⁰, R³¹ and R³² at each occurrence are independently selected from the group consisting of hydrogen, halogen, alkyl, alkenyl, alkynyl, alkoxy, alkenoxy, alkynoxy, thioalkoxy, hydroxyalkyl, aliphatic acyl, —CF₃, —CO₂H, —SH, —OH, —CN, —NO₂, —NH₂, alkynylamino, alkoxycarbonyl, heterocycloyl, carboxy, —N(C₁-C₃ alkyl)-C(O)(C₁-C₃ alkyl), —NHC(O)N(C₁-C₃ alkyl)C(O)NH(C₁-C₃alkyl), —NHC(O)NH(C₁-C₆ alkyl), —NHSO₂(C₁-C₃ alkyl), —NHSO₂(aryl), alkoxyalkyl, alkylamino, alkenylamino, di(C₁-C₃)amino, —C(O)O—(C₁-C₃)alkyl, —C(O)NH—(C₁-C₃)alkyl, —C(O)N(C₁-C₃ alkyl)₂, —CH═NOH, —PO₃H₂, —OPO₃H₂, haloalkyl, alkoxyalkoxy, carboxaldehyde, carboxamide, cycloalkyl, cycloalkenyl, cycloalkynyl, cycloalkylalkyl, aryl, aroyl, aryloxy, arylamino, biaryl, thioaryl, diarylamino, heterocyclyl, alkylaryl, aralkenyl, aralkyl, alkylheterocyclyl, heterocyclylalkyl, sulfonyl, —SO₂—(C₁-C₃ alkyl), —SO₃—(C₁-C₃ alkyl), sulfonamido, carbamate, aryloxyalkyl and —C(O)NH(benzyl) groups; and
R²⁹, at each occurrence, is independently selected from the group consisting of halogen, alkyl, alkenyl, alkynyl, alkoxy, alkenoxy, alkynoxy, thioalkoxy, hydroxyalkyl, aliphatic acyl, —CF₃, —CO₂H, —SH, —CN, —NO₂, —NH₂, —OH, alkynylamino, alkoxycarbonyl, heterocycloyl, carboxy, —N(C₁-C₃ alkyl)-C(O)(C₁-C₃ alkyl), —NHC(O)N(C₁-C₃ alkyl)C(O)NH(C₁-C₃ alkyl), —NHC(O)NH(C₁-C₆ alkyl), —NHSO₂(C₁-C₃ alkyl), —NHSO₂(aryl), alkoxyalkyl, alkylamino, alkenylamino, di(C₁-C₃)amino, —C(O)O—(C₁-C₃)alkyl, —C(O)NH—(C₁-C₃)alkyl, —C(O)N(C₁-C₃ alkyl)₂, —CH═NOH, —PO₃H₂, —OPO₃H₂, haloalkyl, alkoxyalkoxy, carboxaldehyde, carboxamide, cycloalkyl, cycloalkenyl, cycloalkynyl, cycloalkylalkyl, aryl, aroyl, aryloxy, arylamino, biaryl, thioaryl, diarylamino, heterocyclyl, alkylaryl, aralkenyl, aralkyl, alkylheterocyclyl, heterocyclylalkyl, sulfonyl, —SO₂—(C₁-C₃ alkyl), —SO₃—(C₁-C₃ alkyl), sulfonamido, carbamate, aryloxyalkyl and —C(O)NH(benzyl) groups;
wherein B, R⁴, R⁵, R⁶, R⁷, R⁹, R¹⁰, R¹¹, R¹⁸, R²⁹, R³⁰, R³¹, R³² and R³³ are unsubstituted or substituted with at least one electron donating or electron withdrawing group;
wherein when L is NR¹¹, R⁴ and R¹¹ taken together may form a ring;
and wherein R⁹ and R¹⁰ taken together may form a ring;
or a pharmaceutically acceptable salt thereof;
one or more other therapeutically active compounds and a pharmacologically acceptable diluent.

13. The composition of claim 12 wherein z is three or four.

14. The composition of claim 1 where the compound of structure (I) is (3S)-3-[({[1-(2-chlorobenzyl)-4-hydroxy-5-methyl-2-oxo-1,2-dihydropyridin-3-yl]amino}carbonyl)amino]-3-(4-methylphenyl)propanoic acid and pharmaceutically acceptable salts thereof.

15. The composition of claim 1 where the compound of structure (I) is (3S)-3-[({[1-(2-chlorobenzyl)-4-hydroxy-2-oxo-2,5,6,7-tetrahydro-1H-cyclopenta[b]pyridin-3-1]amino}carbonyl)amino]-3-(4-methylphenyl)propanoic acid and pharmaceutically acceptable salts thereof.

16. The composition of claim 1 where the compound of structure (I) is (3S)-3-[({[1-(2-chlorobenzyl)-4-hydroxy-5-methyl-2-oxo- 1,2-dihydropyridin-3-yl]amino}carbonyl)amino]-3-[3-(diethylamino)phenyl]propanoic acid and pharmaceutically acceptable salts thereof.

17. The composition of claim 1 where the compound of structure (I) is (3S)-3-[({[1-(2-chlorobenzyl)-4-hydroxy-2-oxo-2,5,6,7-tetrahydro-1H-cyclopenta[b]pyridin-3-amino}carbonyl)amino]-3-(3-isopropylphenyl)propanoic acid and pharmaceutically acceptable salts thereof.

18. The composition of claim 1 where the compound of structure (I) is (3S)-3-[({[1-(2-chlorobenzyl)-4-hydroxy-2-oxo- 1,2-dihydro-1,8-napthyridin-3-yl]amino}carbonyl)amino-3-(4-methylphenyl)propanoic acid and pharmaceutically acceptable salts thereof.

19. The composition of claim 1 where the other therapeutically active compounds are selected from the group consisting of IL-5 antagonists, CCR-3 antagonists, corticosteroids, antihistamines, Leukotrine antagonists, COX-I and COX-II inhibitors, mast cell stabilizers, anti IL-5 and anti IgE antibodies, IL-5 synthesis and release inhibitors, TNF-α inhibitors, p38 MAP kinase inhibitors, tryptase inhibitors, anticytokine/antichemokine agents, vaccines, cromolyn, selectin antagonists, PDE 4 inhibitors, β-agonists, muscarininc antagonists and immunosuppressives, CD20 antagonists and syk tyrosine kinase inhibitors.

20. A method for treating an inflammatory disease in a mammal comprising administering to said mammal a therapeutically effective amount of a composition of claim 1.

21. The method of claim 20 wherein the inflammatory disease is selected from psoriasis, asthma, atherosclerosis, multiple sclerosis, Guillan-Barr Syndrome, rheumatoid arthritis, inflammatory bowel disease and reperfusion injury.

22. A method for treating an inflammatory disease in a mammal comprising administering to said mammal a therapeutically effective amount of a combination of a compound of structure (I) in claim 1 and an effective amount of one or more other therapeutic compounds.

23. The method of claim 22 wherein the inflammatory disease is selected from psoriasis, asthma, atherosclerosis, multiple sclerosis, Guillan-Barr Syndrome, rheumatoid arthritis, inflammatory bowel disease and reperfusion injury.

24. The composition of claim 19 wherein the compound of structure (I) is selected from the group consisting of (3S)-3-[({[1-(2-chlorobenzyl)-4-hydroxy-5-methyl-2-oxo-1,2-dihydropyridin-3-yl]amino}carbonyl)amino]-3-(4-methylphenyl)propanoic acid; (3S)-3-[({[1-(2-chlorobenzyl)-4-hydroxy-2-oxo-2,5,6,7-tetrahydro-1H-cyclopenta [b]pyridin-3-1]amino}carbonyl)amino]-3-(4-methylphenyl)propanoic acid; (3S)-3-[({[1-(2-chlorobenzyl)-4-hydroxy-5-methyl-2-oxo-1,2-dihydropyridin-3-yl]amino}carbonyl)amino]-3-[3-(diethylamino)phenyl]propanoic acid; (3S)-3-[({[1-(2-chlorobenzyl)-4-hydroxy-2-oxo-2,5,6,7-tetrahydro-1H-cyclopenta[b]pyridin-3-yl]amino}carbonyl)amino]-3-(3-isopropylphenyl)propanoic acid; and (3S)-3-[({[1-(2-chlorobenzyl)-4-hydroxy-2-oxo-1,2-dihydro-1,8-napthyridin-3-yl]amino}carbonyl) amino-3-(4-methylphenyl)propanoic acid and pharmaceutically acceptable salts thereof.

25. The method of claim 20 wherein the compound of structure (I) is selected from the group consisting of (3S)-3-[({[1-(2-chlorobenzyl)-4-hydroxy-5-methyl-2-oxo-1,2-dihydropyridin-3-yl]amino}carbonyl)amino]-3-(4-methylphenyl)propanoic acid; (3S)-3-[({[1-(2-chlorobenzyl)-4-hydroxy-2-oxo-2,5,6,7-tetrahydro-1H-cyclopenta [b]pyridin-3-1]amino}carbonyl)amino]-3-(4-methylphenyl)propanoic acid; (3S)-3-[({[1-(2-chlorobenzyl)-4-hydroxy-5-methyl-2-oxo-1,2-dihydropyridin-3-yl]amino}carbonyl)amino]-3-[3-(diethylamino)phenyl]propanoic acid; (3S)-3-[({[1-(2-chlorobenzyl)-4-hydroxy-2-oxo-2,5,6,7-tetrahydro-1H-cyclopenta[b]pyridin-3-yl]amino}carbonyl)amino]-3-(3-isopropylphenyl)propanoic acid; and (3S)-3-[({[1-(2-chlorobenzyl)-4-hydroxy-2-oxo-1,2-dihydro-1,8-napthyridin-3-yl]amino}carbonyl) amino-3-(4-methylphenyl)propanoic acid and pharmaceutically acceptable salts thereof.

26. A kit comprising in a single package, one container comprising a compound that inhibits binding of an α₄β₁ integrin to its receptors as set forth in structure (I) in claim 1 in a pharmaceutically acceptable carrier and one or more separate containers comprising other therapeutic compounds in pharmaceutically acceptable carriers, with the compound that inhibits binding of α₄β₁ integrin to its receptors and the other therapeutic compounds being present in amounts such that the combination is effective to treat disease states mediated by α₄β₁ integrin binding.

27. The method of claim 20 comprising administering a combination of a compound of claim 1 and beta interferon.

28. The method of claim 27 wherein the compound of claim 1 is (3S)-3-[({[1-(2-chlorobenzyl)-4-hydroxy-2-oxo-2,5,6,7-tetrahydro-1H -cyclopenta[b]pyridine-3-yl]amino}carbonyl)amino]-3-(4-methylphenyl)propanoic acid or a pharmaceutically acceptable salt thereof.

29. The method of claim 3 for treating multiple sclerosis comprising administering a combination of (3S)-3-[({[1-(2-chlorobenzyl)-4-hydroxy-2-oxo-2,5,6,7-tetrahydro-1H-cyclopenta[b]pyridine-3-yl]amino}carbonyl)amino]-3-(4-methylphenyl)propanoic acid or a pharmaceutically acceptable salt thereof and beta interferon.

30. The method of claim 2 comprising administering a combination of a compound of claim 1 and a corticosteroid.

31. The method of claim 30 wherein the compound of claim 1 is (3S)-3-[({[1-(2-chlorobenzyl)-4-hydroxy-2-oxo-2,5,6,7-tetrahydro-1H -cyclopenta[b]pyridine-3-yl]amino}carbonyl)amino]-3-(4-methylphenyl)propanoic acid or a pharmaceutically acceptable salt thereof.

32. The method of claim 30 wherein the compound of claim 1 is (3S)-3-[({[1-(2-chlorobenzyl )-4-hydroxy-2-oxo-2,5,6,7-tetrahydro-1H-cyclopenta[b]pyridine-3-yl]amino}carbonyl)amino]-3-(4-methylphenyl)propanoic acid or a pharmaceutically acceptable salt thereof and the corticosteroid is selected from the group consisting of prednisolone, fluticasone, triamcinolone, beclomethasone, mometasone, budesonide, betamethasone, dexamethasone, prednisone, flunisolide and cortisone.

33. The method of claim 22 comprising administering a combination of a compound of in claim 1 and an immunosuppressant.

34. The method of claim 33 wherein the compound of claim 1 is (3S)-3-[({[1-(2-chlorobenzyl)-4-hydroxy-2-oxo-2,5,6,7-tetrahydro-1H-cyclopenta[b]pyridine-3-yl]amino}-carbonyl)amino]-3-(4-methyphenyl)propanoic acid or a pharmaceutically acceptable salt thereof.

35. The method of claim 22 comprising administering a combination of a compound of claim 1 and a therapeutic compound selected from the group consisting of mycophenolate mofetil, methotrexate, azathioprine and cyclophosphamide.

36. The method of claim 35 wherein the compound claim 1 is (3S)-3-[({[1-(2-chlorobenzyl)-4-hydroxy-2-oxo-2,5,6,7-tetrahydro-1H-cyclopenta[b]pyridine-3-yl]amino}carbonyl)amino]-3-(4-methylphenyl)propanoic acid or a pharmaceutically acceptable salt thereof.
